(19) United States
(12) Patent Application Publication
(10) Pub. No.: US 2003/0087963 A1
(43) Pub. Date: May 8, 2003

(54) Methods of preparing and using 2-hydroxy derivatives of sibutramine and its metabolites

(76) Inventors: Chris H. Senanayake, Shrewsbury, MA (US); Thomas P. Jerussi, Framingham, MA (US); Mark G. Currie, Sterling, MA (US); Qun K. Fang, Wellesley, MA (US); Bob Hsu, Ringoes, NJ (US)

Correspondence Address: PENNIE & EDMONDS LLP, 1667 K STREET NW, SUITE 1000, WASHINGTON, DC, 20006

(21) Appl. No.: 10/238,630

(22) Filed: Sep. 11, 2002

Related U.S. Application Data
(60) Provisional application No. 60/325,192, filed on Sep. 28, 2001.
(60) Provisional application No. 60/318,672, filed on Sep. 13, 2001.

(51) Int. Cl. A61K 31/137; C08B 37/16; C07C 215/28

(52) U.S. Cl. 514/650; 514/58; 564/338; 536/46

(57) ABSTRACT

The invention is directed, in part, to racemic and stereomerically pure 2-hydroxy derivatives of sibutramine and its metabolites, and 2-hydroxy derivatives of desmethylsibutramine and didesmethylsibutramine in particular. The invention is further directed to novel methods of preparing these derivatives are also disclosed.

The invention is also directed to pharmaceutical compositions and dosage forms that comprise therapeutically or prophylactically effective amounts of the compounds, optionally in combination with an additional pharmacologically active compound. These pharmaceutical compositions and dosage forms can be used in the methods of the invention, which provide for the treatment or prevention of a variety of diseases and disorders.

### 1. FIELD OF THE INVENTION

This invention relates to 2-hydroxy derivatives of sibutramine and sibutramine metabolites, which include desmethylsibutramine and didesmethylsibutramine, and to methods of preparing and using the same.

### 2. BACKGROUND OF THE INVENTION

Sibutramine is a neuronal monoamine reuptake inhibitor, which has the chemical name [N-1-[1-(4-chlorophenyl)cyclobutyl]-3-methylbutyl]-N,N-dimethylamine. Originally disclosed in U.S. Pat. Nos. 4,746,680 and 4,806,570, sibutramine inhibits the reuptake of norepinephrine and, to a lesser extent, serotonin and dopamine. See, e.g., Buckett et al., Prog. Neuro-psychopharm. & Biol. Psychiat., 12:575-584, 1988; King et al., J. Clin. Pharm., 26:607-611 (1989).

Racemic sibutramine is sold as a hydrochloride monohydrate under the tradename MERIDIA®, and is indicated for the treatment of obesity. Physician's Desk Reference® 1509-1513 (54^{th} ed., 2000). The treatment of obesity using racemic sibutramine is disclosed, for example, in U.S. Pat. No. 5,436,272.

Sibutramine is rapidly absorbed from the gastrointestinal tract following oral administration and undergoes an extensive first-pass metabolism that yields the metabolites desmethylsibutramine (“DMS”) and didesmethylsibutramine (“DDMS”), as shown below:

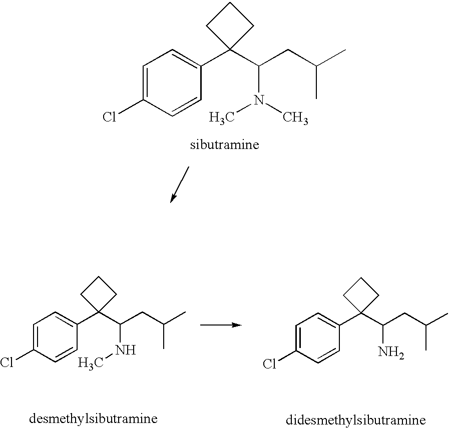

Both didesmethylsibutramine and desmethylsibutramine have interesting and useful biological properties. Each of these sibutramine metabolites can exist as an enantiomeric pair of R and S enantiomers, as shown below, which also exhibit interesting and useful biological properties:

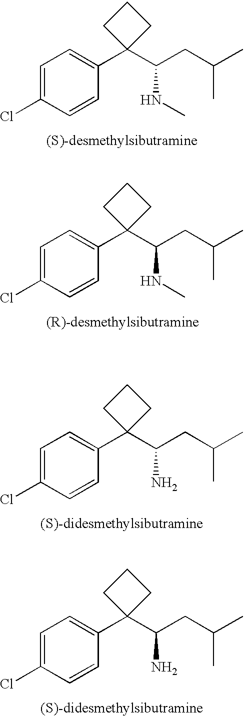

Sibutramine has a variety of adverse effects. See, e.g., Physician's Desk Reference® 1494-1498 (53^{rd} ed., 1999). Coupled with the reported benefits and therapeutic insufficiencies of sibutramine, this fact has encouraged the discovery of compounds and compositions that can be used in the treatment or prevention of disorders such as, but not limited to, sexual (e.g., erectile) dysfunction, affective disorders, weight gain or obesity, cerebral function disorders, pain, obsessive-compulsive disorder, substance abuse, chronic disorders, anxiety, eating disorders, migraines, and incontinence. In particular, compounds and compositions are desired that can be used for the treatment and prevention of such disorders and conditions while incurring fewer of the adverse effects associated with sibutramine.

### 3. SUMMARY OF THE INVENTION

This invention is directed, in part, to racemic and stereomerically pure 2-hydroxy derivatives of sibutramine and sibutramine metabolites, and to 2-hydroxy derivatives of desmethylsibutramine and didesmethylsibutramine in particular. The invention is further directed to novel methods of preparing such compounds.

The invention is also directed to pharmaceutical compositions and dosage forms that comprise therapeutically or prophylactically effective amounts of the compounds, optionally in combination with an additional pharmacologically active compound.

Also encompassed by this invention are methods of treating and preventing diseases and disorders that are ameliorated by the inhibition of neuronal monoamine uptake. Examples of such diseases and disorders include, but are not limited to: eating disorders; weight gain; obesity; irritable bowel syndrome; obsessive-compulsive disorders; platelet adhesion; apnea; affective disorders such as attention deficit disorders, depression, and anxiety; male and female sexual function disorders; restless leg syndrome; osteoarthritis; substance abuse including nicotine and cocaine addiction; narcolepsy; pain such as neuropathic pain, diabetic neuropathy, and chronic pain; migraines; cerebral function disorders; chronic disorders such as premenstrual syndrome; and incontinence.

### 3.1. Definitions

As used herein, the term “prodrug” means a derivative of a compound that can hydrolyze, oxidize, or otherwise react under biological conditions (in vitro or in vivo) to provide the compound. Examples of prodrugs include, but are not limited to, derivatives of 2-hydroxydesmethylsibutramine and 2-hydroxydidesmethylsibutramine that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of 2-hydroxydesmethylsibutramine and 2-hydroxydidesmethylsibutramine that comprise —NO, —NO₂, —ONO, and —ONO₂ moieties.

As used herein, prodrugs of 2-hydroxydidesmethylsibutramine do not include didesmethylsibutramine, desmethylsibutramine, sibutramine, 2-hydroxydesmethylsibutramine, or 2-hydroxysibutramine and prodrugs of 2-hydroxydesmethylsibutramine do not include desmethylsibutramine, sibutramine, 2-hydroxydesmethylsibutramine, or 2-hydroxysibutramine.

As used herein, the terms “biohydrolyzable carbamate,” “biohydrolyzable carbonate,” “biohydrolyzable ureide,” “biohydrolyzable phosphate” mean a carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically less active or inactive but is converted in vivo to the biologically active compound. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein, the term “biohydrolyzable ester” means an ester of a compound that either: 1) does not interfere with the biological activity of the compound but can confer upon that compound advantageous properties in vivo, such as uptake, duration of action, or onset of action; or 2) is biologically less active or inactive but is converted in vivo to the biologically active compound. Examples of biohydrolyzable esters include, but are not limited to, lower alkyl esters, alkoxyacyloxy esters, alkyl acylamino alkyl esters, and choline esters.

As used herein, the term “biohydrolyzable amide” means an amide of a compound that either: 1) does not interfere with the biological activity of the compound but can confer upon that compound advantageous properties in vivo, such as uptake, duration of action, or onset of action; or 2) is biologically less active or inactive but is converted in vivo to the biologically active compound. Examples of biohydrolyzable amides include, but are not limited to, lower alkyl amides, a-amino acid amides, alkoxyacyl amides, and alkylaminoalkylcarbonyl amides.

As used herein, the term “pharmaceutically acceptable salt” refers to a salt prepared from a pharmaceutically acceptable non-toxic inorganic or organic acid. Suitable non-toxic acids include, but are not limited to, acetic, benzenesulfonic, benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, and p-toluenesulfonic acids. For example, specific pharmaceutically acceptable salts are hydrochloride, maleic acid, and tartaric acid salts.

As used herein and unless otherwise indicated, the term “alkyl” includes saturated monovalent linear, branched, and cyclic hydrocarbon radicals. An alkyl group can include one or more double or triple bonds. It is understood that cyclic alkyl groups comprise at least three carbon atoms.

As used herein and unless otherwise indicated, the term “lower alkyl” means branched or linear alkyl having from 1 to 6, more preferably from 1 to 4 carbon atoms. Examples include, but are not limited to, methyl, ethyl, propyl, isopropyl, isobutyl, and tertiary butyl.

As used herein and unless otherwise indicated, the term “aryl” includes an organic radical derived from an aromatic hydrocarbon by removal of one hydrogen, such as phenyl or naphthyl.

As used herein and unless otherwise indicated, the term “aralkyl” means an aryl substituted with one or linear, branched, or cyclic alkyl groups. Aralkyl moieties can be attached to other moieties through their aryl or alkyl components.

As used herein and unless otherwise indicated, the terms “heterocyclic group” and “heterocycle” include aromatic and non-aromatic heterocyclic groups containing one or more heteroatoms each selected from 0, S and N. Non-aromatic heterocyclic groups include groups having only 3 atoms in their ring system, but aromatic heterocyclic groups (i.e., heteroaryl groups) must have at least 5 atoms in their ring system. Heterocyclic groups include benzo-fused ring systems and ring systems substituted with one or more oxo moieties. An example of a 4 membered heterocyclic group is azetidinyl (derived from azetidine). An example of a 5 membered heterocyclic group is thiazolyl, and an example of a 10 membered heterocyclic group is quinolinyl. Examples of non-aromatic heterocyclic groups include, but are not limited to, pyrrolidinyl, tetrahydrofuranyl, tetrahydrothienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, and substituted derivative thereof. Examples of aromatic heterocyclic groups include, but are not limited to, pyridinyl, methylpyridine analgoues, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzoimidazoles, benzofuranyl, cinnolinyl, indazolyl, indolinyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, furopyridinyl, and substituted derivatives thereof. The foregoing groups, as derived from the compounds listed above, may be C-attached or N-attached where such attachment is possible. For instance, a group derived from pyrrole can be pyrrol-1-yl (N-attached) or pyrrol-3-yl (C-attached).

As used herein and unless otherwise indicated, the term “heteroaryl” means an aromatic heterocycle.

As used herein and unless otherwise indicated, the term “substituted” as used to describe a compound or chemical moiety means that at least one hydrogen atom of that compound or chemical moiety is replaced with a second chemical moiety. Examples of second chemical moieties include, but are not limited to: halogen atoms (e.g., chlorine, bromine, and iodine); C₁-C₆ linear, branched, or cyclic alkyl (e.g., methyl, ethyl, butyl, tert-butyl, and cyclobutyl); hydroxyl; thiols; carboxylic acids; esters, amides, silanes, nitriles, thioethers, stannanes, and primary, secondary, and tertiary amines (e.g., —NH₂, —NH(CH₃), —N(CH₃)₂, and cyclic amines). Preferred second chemical moieties are chlorine, hydroxyl, methoxy, amine, thiol, and carboxylic acid.

As used herein and unless otherwise indicated, a composition that is “substantially free” of a compound means that the composition contains less than about 20% by weight, more preferably less than about 10% by weight, even more preferably less than about 5% by weight, and most preferably less than about 3% by weight of the compound.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of stereoisomer of the compound and less than about 20% by weight of other stereoisomers the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

As used herein and unless otherwise indicated, the term “enantiomerically pure” means a stereomerically pure composition of a compound having one chiral center.

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

### 4. DETAILED DESCRIPTION OF THE INVENTION

This invention is directed to compounds of Formula 1:

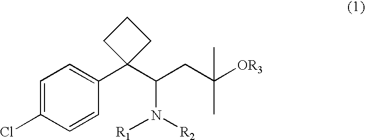

wherein each of R₁ and R₂ is independently hydrogen, substituted or unsubstituted alkyl, substituted or unsubstituted aryl, substituted or unsubstituted aralkyl, ketone, ester, or amide, and R₃ is hydrogen, substituted or unsubstituted alkyl, substituted or unsubstituted aryl, substituted or unsubstituted aralkyl, and to pharmaceutically acceptable salts, solvates, hydrate, clathrates, and prodrugs thereof. The invention is further directed to methods of preparing such compounds, to pharmaceutical compositions and dosage forms comprising them, and to methods of their use. In preferred compounds of Formula (1), Preferred compounds of the invention are 2-hydroxy derivatives of sibutramine and sibutramine metabolites, and pharmaceutically acceptable salts, solvates, hydrate, clathrates, and prodrugs thereof. The invention encompasses stereomerically pure forms of chiral compounds of the invention as well as mixtures (e.g., racemic mixtures) of two or more stereomerically pure forms of the compounds.

A first embodiment of the invention encompasses 2-hydroxysibutramine and pharmaceutically acceptable salts, solvates, hydrate, clathrates, and prodrugs thereof. As shown below, 2-hydroxysibutramine can be racemic or enantiomerically pure:

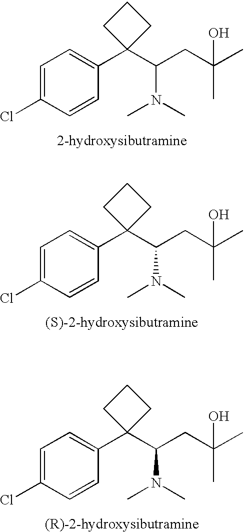

A second embodiment of the invention encompasses 2-hydroxy derivatives of sibutramine metabolites such as, but not limited to, desmethylsibutramine and didesmethylsibutramine, and pharmaceutically acceptable salts, solvates, hydrate, clathrates, and prodrugs thereof. As shown below, 2-hydroxydesmethylsibutramine can be racemic or enantiomerically pure:

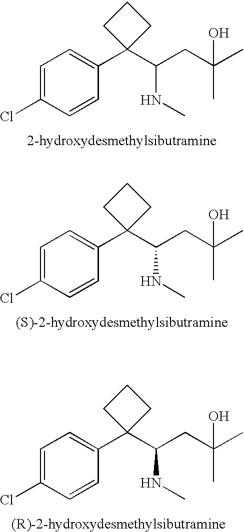

(S)-2-hydroxydesmethylsibutramine (R)-2-hydroxydesmethylsibutramine

As shown below, 2-hydroxydidesmethylsibutramine can also be racemic or enantiomerically pure:

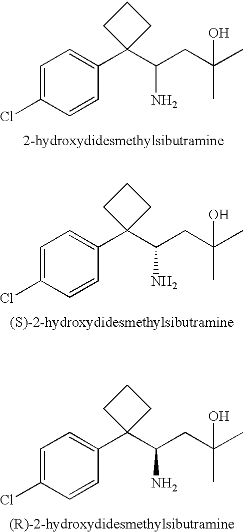

A third embodiment of the invention encompasses a method of preparing 2-hydroxydidesmethylsibutramine, or a pharmaceutically acceptable salt, solvate, clathrate, hydrate, or prodrug thereof, which comprises contacting a compound of Formula 2:

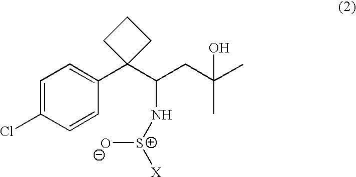

wherein X is substituted or unsubstituted alkyl, substituted or unsubstituted aralkyl, or substituted or unsubstituted aryl, with a reagent capable of cleaving a nitrogen-sulfur bond under conditions suitable for the formation of 2-hydroxydidesmethylsibutramine. In a preferred method, the 2-hydroxydidesmethylsibutramine is enantiomerically pure.

A fourth embodiment of the invention encompasses a method of preparing 2-hydroxydesmethylsibutramine, or a pharmaceutically acceptable salt, solvate, clathrate, hydrate, or prodrug thereof, which comprises contacting a compound of Formula 2 with a reagent capable of cleaving a nitrogen-sulfur bond under conditions suitable for the formation of 2-hydroxydidesmethylsibutramine, and contacting the 2-hydroxydidesmethylsibutramine with a methylating reagent under conditions sufficient for the formation of 2-hydroxydesmethylsibutramine. In a preferred method, the 2-hydroxydesmethylsibutramine is enantiomerically pure.

In a preferred method of each of the third and fourth embodiments, the compound of Formula 2 is stereomerically pure. In another preferred method, the 2-hydroxydesmethylsibutramine or 2-hydroxydidesmethylsibutramine is provided as a pharmaceutically acceptable salt. Examples of preferred pharmaceutically acceptable salts include, but are not limited to, acetic, benzenesulfonic, benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, and p-toluenesulfonic salts.

In another preferred method of each of the third and fourth embodiments, the reagent capable of cleaving a nitrogen-sulfur bond is an acid. A preferred acid is HCl.

The compound of Formula 2 can be prepared by contacting a compound of Formula

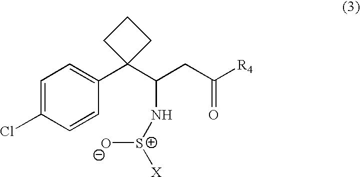

wherein R₄ is a substituted or unsubstituted alkyl (e.g., methyl, ethyl, or t-butyl), aryl, —OR₅, or —SR₅, wherein R₅ is alkyl, aryl, or aralkyl, with an alkylating agent under conditions sufficient for the formation of the compound of Formula 2. Preferably, R₄ is methyl. Preferred alkylating agents include, but are not limited to, Grignard reagents, (e.g., MeMgHal, wherein Hal is a halogen such as Br), MeLi, Me₂CuLi, MeMgBr/CeCl₃, and MeLi/CeCl₃. Preferably, the compound of Formula 3 is stereomerically pure.

The compound of Formula 3 is preferably prepared by contacting a compound of Formula 4:

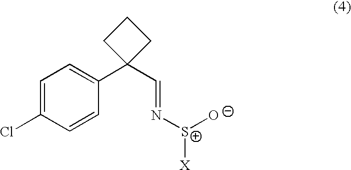

with the compound of Formula 5:

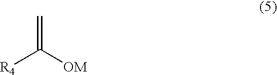

wherein R₄ is defined above and M is a metal (e.g., Li, Mg, or a complex of Ti) under conditions sufficient for the formation of the compound of Formula 3. A preferred compound of Formula 5 is of the formula:

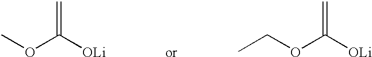

optionally in combination with Ti(i-PrO)₃Cl.

The compound of Formula 4 can be prepared by contacting the compound of Formula 6:

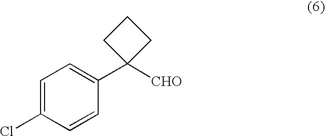

with a compound of Formula 7:

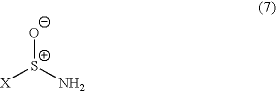

wherein X is defined above, under conditions suitable for the formation of the compound of Formula 4.

Preferably, the compound of Formula 7 is stereomerically pure, as shown below:

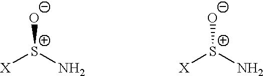

Specific compounds of Formula 7 include, but are not limited to, (R)-tert-butylsulfinamide and (S)-tert-butylsulfinamide.

A fifth embodiment of the invention encompasses a method of preparing 2-hydroxydidesmethylsibutramine, or a pharmaceutically acceptable salt, solvate, clathrate, hydrate, or prodrug thereof, which comprises contacting a compound of Formula 3 with a reagent capable of cleaving a nitrogen-sulfur bond under conditions suitable for the formation of the compound of Formula 8:

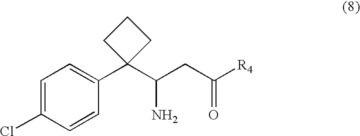

and contacting the compound of Formula 8 with an alkylating agent under conditions suitable for the formation of 2-hydroxydidesmethylsibutramine. In a preferred method, the 2-hydroxydidesmethylsibutramine is enantiomerically pure. Preferred alkylating agents include, but are not limited to, Grignard reagents, (e.g., MeMgHal, wherein Hal is a halogen such as Br), MeLi, Me₂CuLi, MeMgBr/CeCl₃, and MeLi/CeCl₃.

A sixth embodiment of the invention encompasses a method of preparing 2-hydroxydesmethylsibutramine, or a pharmaceutically acceptable salt, solvate, clathrate, hydrate, or prodrug thereof, which comprises contacting a compound of Formula 3 with an alkylating agent under conditions suitable for the formation of a compound of Formula 2, and contacting the compound of Formula 2 with a reagent capable of cleaving a nitrogen-sulfur bond under conditions suitable for the formation of 2-hydroxydidesmethylsibutramine, and contacting the 2-hydroxydidesmethylsibutramine with a methylating reagent under conditions sufficient for the formation of 2-hydroxydesmethylsibutramine. In a preferred method, the 2-hydroxydesmethylsibutramine is enantiomerically pure.

A seventh embodiment of the invention encompasses a method of treating or preventing a sexual function disorder, which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof, optionally in combination with a 5-HT₃ antagonist. Preferred 2-hydroxy derivatives include, but are not limited to, 2-hydroxydesmethylsibutramine and 2-hydroxydidesmethylsibutramine. Preferred 2-hydroxysibutramine and 2-hydroxy derivatives of sibutramine metabolites are stereomerically pure. A particular 2-hydroxy derivative is enantiomerically pure (R)-didesmethylsibutramine.

In a preferred method of this embodiment, the 2-hydroxysibutramine or a 2-hydroxy derivative of a sibutramine metabolite, or pharmaceutically acceptable salt, solvate, hydrate, clathrate, or prodrug thereof, is administered to the patient orally, transdermally, or mucosally.

In another preferred method of this embodiment, the patient in need of treatment or prevention is elderly or postmenstrual.

As used herein, the terms “sexual dysfunction” and “sexual function disorder” encompass sexual dysfunction in men and women caused by psychological and/or physiological factors. Examples of sexual dysfunction include, but are not limited to, erectile dysfunction, vaginal dryness, lack of sexual excitement, or inability to obtain orgasm. The term “sexual dysfunction” further encompasses psycho-sexual dysfunction. Examples of psycho-sexual dysfunction include, but are not limited to, inhibited sexual desire, inhibited sexual excitement, inhibited female orgasm, inhibited male orgasm, premature ejaculation, functional dysparcunia, functional vaginismus, and a typical psychosexual dysfunction.

An eighth embodiment of the invention encompasses a method of treating or preventing an affective disorder which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof. Preferred 2-hydroxy derivatives include, but are not limited to, 2-hydroxydesmethylsibutramine and 2-hydroxydidesmethylsibutramine. Preferred 2-hydroxysibutramine and 2-hydroxy derivatives of sibutramine metabolites are stereomerically pure. A particular 2-hydroxy derivative is enantiomerically pure (R)-didesmethylsibutramine.

Affective disorders include, but are not limited to, depression (e.g., melancholia), attention deficit disorder (including attention deficit disorder with hyperactivity and attention deficit/hyperactivity disorder), bipolar and manic conditions, dysthymic disorder, and cyclothymic disorder. As used herein, the terms “attention deficit disorder” (ADD), “attention deficit disorder with hyperactivity” (ADDH), and “attention deficit/hyperactivity disorder” (AD/HD), are used in accordance with their accepted meanings in the art. See, e.g., Diagnostic and Statistical Manual of Mental Disorders, Fourth Ed., American Psychiatric Association, 1997 (DSM-IV™) and Diagnostic and Statistical Manual of Mental Disorders, 3^{rd} Ed., American Psychiatric Association (1981) (DSM-III™).

A preferred method of this embodiment is a method of treating or preventing attention deficit disorder. In a particular embodiment, the patient is a child (e.g., aged 3-18 years).

Another preferred method of this embodiment is a method of treating or preventing depression. As used herein, the term “treating or preventing depression” means relief from or prevention of the symptoms of depression which include, but are not limited to, changes in mood, feelings of intense sadness, despair, mental slowing, loss of concentration, pessimistic worry, agitation, and self-deprecation. Physical changes can also be relieved or prevented by this method, and include, but are not limited to, insomnia, anorexia, decreased energy and libido, and abnormal hormonal circadian rhythms.

A ninth embodiment of the invention encompasses a method of treating or preventing weight gain or obesity which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof, optionally in combination with a lipase inhibitor. Preferred 2-hydroxy derivatives include, but are not limited to, 2-hydroxydesmethylsibutramine and 2-hydroxydidesmethylsibutrarnine. Preferred 2-hydroxysibutramine and 2-hydroxy derivatives of sibutramine metabolites are stereomerically pure. A particular 2-hydroxy derivative is enantiomerically pure (R)-didesmethylsibutramine.

As used herein, the term “treating or preventing weight gain or obesity” means reduction of weight, relief from being overweight, treating weight gain caused by the administration of other drugs, relief from gaining weight, or relief from obesity, and prevention from gaining weight, all of which are usually due to unnecessary consumption of food. The invention also encompasses methods of treating or preventing conditions incidental to obesity including, but not limited to, hypertension, such as pulmonary hypertension; cancers, such as breast, colon, gall bladder, and endometrial; gall stones; cardiovascular disease, such as dyslipidemia and carotid intimal medial thickening; hiatial hernia; osteoarthritis; gout; thyroid disease, such as diabetes; gastro-esophogeal reflux disease; menstrual dysfunction; and infertility.

In a particular method of this embodiment, the weight gain is associated with the administration of a drug that induces weight gain. In another method of this embodiment, the weight gain is associated with smoking cessation.

A tenth embodiment encompasses a method of treating or preventing a disorder associated with the administration of a lipase inhibitor for obesity or weight management, such as, for example, orlistat (XENICAL®), which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof. Preferred 2-hydroxy derivatives include, but are not limited to, 2-hydroxydesmethylsibutramine and 2-hydroxydidesmethylsibutramine. Preferred 2-hydroxysibutramine and 2-hydroxy derivatives of sibutramine metabolites are stereomerically pure. A particular 2-hydroxy derivative is enantiomerically pure (R)-didesmethylsibutramine.

As used herein, the term “treating or preventing a disorder associated with the administration of a lipase inhibitor” means alleviating or reducing adverse effects associated with administration of a lipase inhibitor, which include, but are not limited to, infectious diarrhea, oily fecal spotting, flatus with discharge, fecal urgency, fatty/oily stool, oily evacuation, increased defecation, anal leakage, and fecal incontinence.

An eleventh embodiment of the invention encompasses a method of treating or preventing a cerebral function disorder which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof. Preferred 2-hydroxy derivatives include, but are not limited to, 2-hydroxydesmethylsibutramine and 2-hydroxydidesmethylsibutramine. Preferred 2-hydroxysibutramine and 2-hydroxy derivatives of sibutramine metabolites are stereomerically pure. A particular 2-hydroxy derivative is enantiomerically pure (R)-didesmethylsibutramine.

Cerebral function disorders include, but are not limited to, senile dementia, Alzheimer's type dementia, memory loss, amnesia/amnestic syndrome, disturbance of consciousness, coma, lowering of attention, speech disorders, Parkinson's disease, Lennox syndrome, autism, epilepsy, hyperkinetic syndrome, and schizophrenia. Cerebral function disorders can be induced by factors including, but not limited to, cerebrovascular diseases, such as cerebral infarction, cerebral bleeding, cerebral arteriosclerosis, cerebral venous thrombosis, and head injuries, and conditions having symptoms selected from the group consisting of disturbances of consciousness, senile dementia, coma, lowering of attention, and speech disorders. As used herein, the term “treating or preventing a cerebral function disorder” means relief from or prevention of one or more symptoms associated with cerebral function disorders.

A twelfth embodiment encompasses a method of treating or preventing restless leg syndrome, which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof. Preferred 2-hydroxy derivatives include, but are not limited to, 2-hydroxydesmethylsibutramine and 2-hydroxydidesmethylsibutramine. Preferred 2-hydroxysibutramine and 2-hydroxy derivatives of sibutramine metabolites are stereomerically pure. A particular 2-hydroxy derivative is enantiomerically pure (R)-didesmethylsibutramine.

In a preferred method, the patient is at least about 50, 60, or 70 years of age. In another preferred method, the 2-hydroxysibutramine or a 2-hydroxy derivative of a sibutramine metabolite is administered in combination with at least one of pergolide, carbidopa, levodopa, oxycodone, carbamazepine, gabapentin, or pharmaceutically acceptable salts, solvates, hydrates, clathrates, prodrugs, optically and pharmacologically active stereoisomers, or pharmacologically active metabolites thereof.

As used herein, the term “restless leg syndrome” encompasses a disorder that typically occurs during sleep or rest, or just before sleep or rest, and which is characterized by uncomfortable sensations in the legs. The disorder often occurs in patients older than about 50 years of age. Examples of uncomfortable sensations in the legs include, but are not limited to, pulling, drawing, crawling, wormy, boring, tingling, pins and needles, prickly and sometimes painful sensations that are usually accompanied by an overwhelming urge to move the legs. As used herein, the term “restless leg syndrome” also encompasses Ekbom Syndrome, Wittmaack-Ecbom Syndrome, Hereditary Acromelalgia, and Anxieties Tibialis.

A thirteenth embodiment of the invention encompasses a method of treating or preventing pain which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof. Preferred 2-hydroxy derivatives include, but are not limited to, 2-hydroxydesmethylsibutramine and 2-hydroxydidesmethylsibutramine. Preferred 2-hydroxysibutramine and 2-hydroxy derivatives of sibutramine metabolites are stereomerically pure. A particular 2-hydroxy derivative is enantiomerically pure (R)-didesmethylsibutramine. In a particular embodiment, the pain is chronic pain, such as neuropathic pain and diabetic neuropathy.

A fourteenth embodiment of the invention encompasses a method of treating or preventing obsessive-compulsive disorder which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof. Preferred 2-hydroxy derivatives include, but are not limited to, 2-hydroxydesmethylsibutramine and 2-hydroxydidesmethylsibutramine. Preferred 2-hydroxysibutramine and 2-hydroxy derivatives of sibutramine metabolites are stereomerically pure. A particular 2-hydroxy derivative is enantiomerically pure (R)-didesmethylsibutramine.

As used herein, the terms “obsessive-compulsive disorder,” “pre-menstrual syndrome,” “anxiety,” and “eating disorder” are used consistently with their accepted meanings in the art. See, e.g., DSM-IV™ and DSM-II™. The term “methods of treating or preventing” when used in connection with these disorders means the amelioration, prevention, or relief from symptoms and/or effects associated with these disorders.

A fifteenth embodiment encompasses a method of treating or preventing substance abuse which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof. Preferred 2-hydroxy derivatives include, but are not limited to, 2-hydroxydesmethylsibutramine and 2-hydroxydidesmethylsibutramine. Preferred 2-hydroxysibutramine and 2-hydroxy derivatives of sibutramine metabolites are stereomerically pure. A particular 2-hydroxy derivative is enantiomerically pure (R)-didesmethylsibutramine. In a particular embodiment, the substance abuse is cocaine addiction or alcohol addiction.

As used herein, the term “substance abuse” encompasses the abuse of, and physical and/or psychological addiction to, drugs or alcohol. The term “substance abuse” further encompasses its accepted meaning in the art. See, e.g., DSM-IV™ and DSM-III™. A preferred method encompassed by this embodiment is a method of treating or preventing cocaine and/or heroin abuse.

A sixteenth embodiment encompasses a method of treating or preventing nicotine addiction which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof. Preferred 2-hydroxy derivatives include, but are not limited to, 2-hydroxydesmethylsibutramine and 2-hydroxydidesmethylsibutramine. Preferred 2-hydroxysibutramine and 2-hydroxy derivatives of sibutramine metabolites are stereomerically pure. A particular 2-hydroxy derivative is enantiomerically pure (R)-didesmethylsibutramine. Nicotine addiction includes nicotine addiction of all known forms, such as addiction to cigarettes, cigars and/or pipes, and chewing tobacco.

A seventeenth embodiment of the inveniton encompasses a method of eliciting smoking cessation which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof. Preferred 2-hydroxy derivatives include, but are not limited to, 2-hydroxydesmethylsibutramine and 2-hydroxydidesmethylsibutramine. Preferred 2-hydroxysibutramine and 2-hydroxy derivatives of sibutramine metabolites are stereomerically pure. A particular 2-hydroxy derivative is enantiomerically pure (R)-didesmethylsibutramine.

In a preferred method, the 2-hydroxysibutramine or a 2-hydroxy derivative of a sibutramine metabolite, or pharmaceutically acceptable salt, solvate, hydrate, clathrate, or prodrug thereof, is administered orally, mucosally, or transdermally. In a more preferred method, it is administered transdermally.

In another preferred method of this embodiment, the 2-hydroxysibutramine or a 2-hydroxy derivative of a sibutramine metabolite, or a pharmaceutically acceptable salt, solvate, hydrate, clathrate, or prodrug thereof, is administered in combination with a therapeutically or prophylactically effective amount of nicotine. Preferably, these compounds are administered orally, mucosally, or transdermally. More preferably, they are administered transdermally.

An eighteenth embodiment of this invention encompasses a method of treating or preventing a chronic disorder, which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof. Preferred 2-hydroxy derivatives include, but are not limited to, 2-hydroxydesmethylsibutramine and 2-hydroxydidesmethylsibutramine. Preferred 2-hydroxysibutramine and 2-hydroxy derivatives of sibutramine metabolites are stereomerically pure. A particular 2-hydroxy derivative is enantiomerically pure (R)-didesmethylsibutramine. Examples of chronic disorders include, but are not limited to, narcolepsy, chronic fatigue syndrome, seasonal affective disorder, fibromyalgia, and premenstrual syndrome (or premenstrual dysphoric disorder), perimenopause, and menopause).

As used herein, the phrases “treatment or prevention of premenstrual syndrome,” “treatment or prevention of perimenopause,” and “treatment or prevention of menopause” mean the reduction or relief from one or more psychological and/or physiological symptoms of the named condition. Examples of such symptoms include, but are not limited to, fatigue, irritability, insomnia, inability to concentrate, depression, memory loss, headache, anxiety, and nervousness.

A nineteenth embodiment encompasses a method of treating or preventing anxiety which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof. Preferred 2-hydroxy derivatives include, but are not limited to, 2-hydroxydesmethylsibutramine and 2-hydroxydidesmethylsibutramine. Preferred 2-hydroxysibutramine and 2-hydroxy derivatives of sibutramine metabolites are stereomerically pure. A particular 2-hydroxy derivative is enantiomerically pure (R)-didesmethylsibutramine.

A twentieth embodiment encompasses a method of treating or preventing an eating disorder including, but not limited to, anorexia, bulimia, binging, and snacking, which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof. Preferred 2-hydroxy derivatives include, but are not limited to, 2-hydroxydesmethylsibutramine and 2-hydroxydidesmethylsibutramine. Preferred 2-hydroxysibutramine and 2-hydroxy derivatives of sibutramine metabolites are stereomerically pure. A particular 2-hydroxy derivative is enantiomerically pure (R)-didesmethylsibutramine.

A twenty first embodiment encompasses a method of treating or preventing migraines which comprises administering to a patient in need thereof a therapeutically or prophylactically effective amount of 2-hydroxysibutrarnine or a 2-hydroxy derivative of a sibutramine metabolite, or a pharmaceutically acceptable salt, solvate, hydrate, clathrate, or prodrug thereof. Preferred 2-hydroxy derivatives include, but are not limited to, 2-hydroxydesmethylsibutramine and 2-hydroxydidesmethylsibutramine. Preferred 2-hydroxysibutramine and 2-hydroxy derivatives of sibutramine metabolites are stereomerically pure. A particular 2-hydroxy derivative is enantiomerically pure (R)-didesmethylsibutramine.

A twenty second embodiment encompasses a method of treating or preventing incontinence which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof. Preferred 2-hydroxy derivatives include, but are not limited to, 2-hydroxydesmethylsibutramine and 2-hydroxydidesmethylsibutramine. Preferred 2-hydroxysibutramine and 2-hydroxy derivatives of sibutramine metabolites are stereomerically pure. A particular 2-hydroxy derivative is enantiomerically pure (R)-didesmethylsibutramine. Particular types of incontinence that can be treated by methods of this embodiment include, but are not limited to, fecal incontinence, stress urinary incontinence (“SUI”), urinary exertional incontinence, urge incontinence, reflex incontinence, passive incontinence, anal leakage, and overflow incontinence.

As used herein, the term “treating or preventing incontinence” means treatment, prevention of, or relief from the symptoms of incontinence including involuntary voiding of feces or urine, and dribbling or leakage or feces or urine, which may be due to one or more causes including, but not limited to, pathology altering sphincter control, loss of cognitive function, overdistention of the bladder, hyper-reflexia and/or involuntary urethral relaxation, weakness of the muscles associated with the bladder or neurologic abnormalities.

A preferred method encompassed by this embodiment is a method of treating or preventing stress urinary incontinence. In a further preferred method encompassed by this embodiment, the patient is an elder human of an age greater than about 50 or a child of an age less than about 13.

In particular methods of each of the embodiments of this invention that are directed to the treatment or prevention of a disease or condition, the therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof, is administered to a patient in combination with an additional pharmacologically active compound. Examples of additional pharmacologically active compounds include, but are not limited to, drugs that act on the central nervous system (“CNS”), such as, but not limited to: 5-HT (e.g., 5-HT₃ and 5-HT_{1A}) agonists and antagonists; selective serotonin reuptake inhibitors (“SSRIs”); hypnotics and sedatives; drugs useful in treating psychiatric disorders including antipsychotic and neuroleptic drugs, antianxiety drugs, antidepressants, and mood-stabilizers; CNS stimulants such as amphetamines; dopamine receptor agonists; antimonic agents; antipanic agents; cardiovascular agents (e.g., beta blockers and angiotensin converting enzyme inhibitors); phosphodiesterase inhibitors; antivirals; antibiotics; antifungals; and antineoplastics. As discussed in more detail herein, the particular additional pharmacologically active compound used in a method will depend upon the disease or condition being treated or prevented, as well as the particular patient being treated.

A final embodiment of the invention encompasses pharmaceutical compositions and single unit dosage forms that comprise a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof. Preferred 2-hydroxy derivatives include, but are not limited to, 2-hydroxydesmethylsibutramine and 2-hydroxydidesmethylsibutramine. Preferred 2-hydroxysibutramine and 2-hydroxy derivatives of sibutramine metabolites are stereomerically pure. Particular 2-hydroxy derivatives include enantiomerically pure (R)-didesmethylsibutramine and (S)-didesmethylsibutramine. Particular pharmaceutical compositions and single unit dosage forms of the invention further comprise an additional pharmacologically active compound.

### 4.1. Preparation of 2-Hydroxy Derivatives of Sibutramine and its Metabolites

Racemic and enantiomerically pure 2-hydroxysibutramine, 2-hydroxydesmethylsibutramine, and 2-hydroxydidesmethylsibutramine can be readily prepared according to the method shown below in Scheme I. This scheme, like others disclosed herein, is merely representative of a method of the invention, and is not to be construed as limiting its scope in any way. For example, although Scheme I shows the stereoselective synthesis of (S)-2-hydroxydidesmethylsibutramine, those of skill in the art will recognize that the scheme can easily be modified to produce the compound's (R) enantiomer, as well as the racemate.

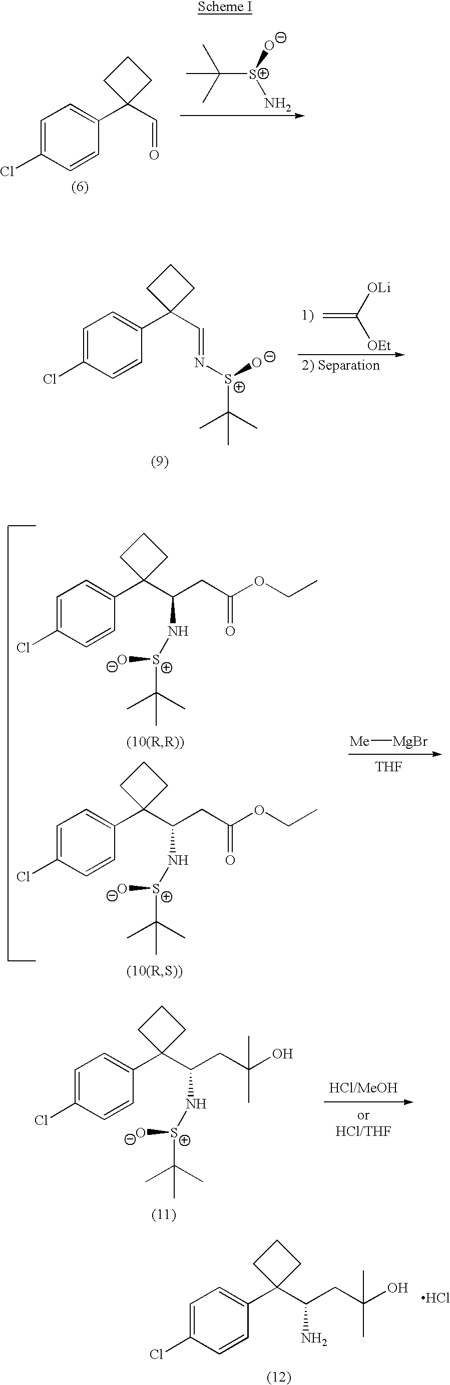

According to this method, 1-(4-chlorophenyl)-cyclobutanecarboxaldehyde (compound 6) is contacted with a sulfinamide under reaction conditions suitable for the formation of compound 9. The compound of Formula 6 can be prepared by, for example, reducing 1-(4-chlorophenyl)-cyclobutane-carbonitrile with a suitable reductant such as, but not limited to, diisobutylaluminum hydride (DIBAL), Red-Al®, or Raney® nickel.

Sulfinamides suitable for use in this invention can be prepared according to the methods known in the art or those disclosed by copending U.S. Provisional Patent Application No. 60/283,337 to Senanayake et al., entitled “Methods of Preparing Sulfinamides and Sulfoxides” and filed Apr. 13, 2001, the entirety of which is incorporated herein by reference. Preferred sulfinamides are stereomerically pure. A preferred sulfinamide is tert-butanesulfinamide (which is also referred to as “tert-butylsulfinamide”) (“TBSA”).

The reaction of compound 6 and the sulfinamide is preferably done in a solvent such as, but not limited to, toluene, THF, CH₂Cl₂, diethyl ether, MTBE, and mixtures thereof. The reaction is preferably catalyzed with a suitable dehydrating agent such as, but not limited to, Ti(alkoxy)₄ (e.g., Ti(OEt)₄ and Ti(O-i-Pr)₄), TiHalₖ(O-i-Pr)₄₋ₖ (wherein Hal is F, Cl, Br, or I, and k is 1, 2, or 3), SnCl₄, MgSO₄, CuSO₄, Na₂SO₄. Preferably, this reaction is run using a ratio of about 1 to about 4 equivalents of compound 5 to about 0.75 to about 1.5 equivalents of sulfinamide and about 0.1 to about 10 equivalents dehydrating agent. The reaction can be run at a temperature of from about −20° C. to about 110° C., more preferably from about 0° C. to about 40° C., and most preferably from about 15° C. to about 25° C.

The imine of Formula 9 is then contacted with ethyl acetate enolate, which can be generated, for example, using lithium bis(trimethylsilyl)amide (“LHMDS”) to yield the compound of Formula 10. As shown in Scheme I, this reaction typically yields a mixture of diastereomers. In the particular reaction shown in Scheme I, the diastereomers are formed in a ratio of about 2:1 10(R,S) to 10(R,R) with a yield of about 84 percent. Flash chromatography can be used to separate them.

In the non-limiting method shown in Scheme I, the major diastereomer 10(R,S) is reacted with excess methyl Grignard or another suitable reagent (e.g., MeLi or Me₂CuLi in optional combination with CeCl₃) to give the alcohol 11. The corresponding ketone is a typical impurity, which can be removed by chromatography. Compound 11 is then contacted with an acid (e.g., 5 N HCl in methanol) to provide the final product, (S)-(+)-2-hydroxydidesmethylsibutramine HCl salt, from which other salts can be readily prepared using standard techniques.

As those of skill in the art will recognize, the order in which various steps of the method shown in Scheme I can be altered in a way that still yields the desired product. For example, the sulfur-based moiety of compound 10 can be cleaved to yield an intermediate, which can be isolated or can be converted in situ to yield the final product.

As those of skill in the art will also recognize, the use of a sulfinamide of the opposite stereochemistry as that shown in Scheme I can be used to provide enantiomerically pure (R)-(−)-2-hydroxydidesmethylsibutramine. Furthermore, the minor isomer of 10(R,R) produced by the method shown in Scheme I can be re-crystallized and subjected to the same reactions described above to provide (R)-(−)-2-hydroxydidesmethyl-sibutramine. In this approach, the tertiary alcohol formed by reaction with the Grignard contains a methyl ketone intermediate that may be difficult to separate. Consequently, the reaction mixture is preferably converted to a mixture of alcohols by contacting it with a reductant such as sodium borohydride, and then purified by chromatography to yield the enantiomerically pure final product.

Racemic and enantiomerically pure 2-hydroxydesmethylsibutramine can be readily prepared from 2-hydroxydidesmethylsibutramine as shown in Scheme II:

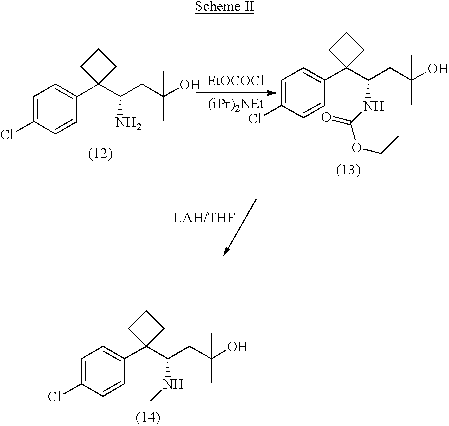

Scheme II shows the preparation of (S)-(−)-2-hydroxydesmethylsibutramine (compound 14) from the corresponding (S)-(+)-2-hydroxydidesmethylsibutramine (compound 12), although the same approach can be used to prepare (R)-(+)-2-hydroxydesmethylsibutramine and racemic 2-hydroxydesmethylsibutramine from the corresponding form of 2-hydroxydidesmethylsibutramine.

In this approach, 2-hydroxydidesmethylsibutramine is contacted with ethylchloroformate in the presence of diisopropylethylamine to give the corresponding ethyl carbamate of Formula 13. The carbamate is then treated with LAH in a suitable solvent such as THF at about 65° C. to give 2-hydroxydesmethylsibutramine, which can be purified by chromatography and converted to a salt if desired. This same procedure can be used to yield racemic and enantiomerically pure 2-hydroxysibutramine.

Using compounds and conditions well known to those of skill in the art, racemic and stereomerically pure compounds of Formula 1, and pharmaceutically acceptable salts, solvates, hydrates, clathrates, and prodrugs thereof, can be readily prepared from intermediates and final products of the synthetic methods of this invention (e.g., those shown in schemes I and H):

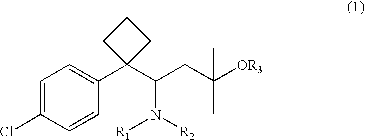

wherein R₁, R₂, and R₃ are defined herein.

### 4.2. Methods of Treatment and Prevention

In each of the methods of the invention, a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof, is administered to a patient. Preferred 2-hydroxy derivatives of sibutramine and sibutramine metabolites are stereomerically pure.

In specific methods of the invention, the 2-hydroxy derivative of sibutramine or a sibutramine metabolite, or pharmaceutically acceptable salt, solvate, hydrate, clathrate, or prodrug thereof, is administered to a patient in an amount from about 0.1 mg to about 60 mg, preferably from about 2 mg to about 30 mg, and more preferably from about 5 mg to about 15 mg. Such amounts can be administered daily as needed for the treatment of acute and chronic diseases and conditions.

Optionally, the 2-hydroxy derivative of sibutramine or a sibutramine metabolite is adjunctively administered (i.e., administered in combination) with one or more additional pharmacologically active compounds. In other words, a 2-hydroxy derivative of sibutramine or a sibutramine metabolite and an additional pharmacologically active compound can be administered to a patient as a combination, concurrently but separately, or sequentially by any suitable route. Suitable routes of administration include oral, mucosal (e.g., nasal, sublingual, buccal, rectal, and vaginal), parenteral (e.g., intravenous, intramuscular or subcutaneous), and transdermal routes.

As physicians and those skilled in the art of pharmacology will readily appreciate, the particular additional pharmacologically active compounds that can be administered in combination with a 2-hydroxy derivative of a sibutramine metabolite will depend on the particular disease or condition being treated or prevented, and may also depend on the age and health of the patient to which the compounds are to be administered.

Additional pharmacologically active compounds that can be used in the methods and compositions of the invention include, but are not limited to, drugs that act on the central nervous system (“CNS”), such as, but not limited to: 5-HT (e.g., 5-HT₃ and 5-HT_{1A}) agonists and antagonists; selective serotonin reuptake inhibitors (“SSRIs”); hypnotics and sedatives; drugs useful in treating psychiatric disorders including antipsychotic and neuroleptic drugs, antianxiety drugs, antidepressants, and mood-stabilizers; CNS stimulants such as amphetamines; dopamine receptor agonists; antimonic agents; antipanic agents; cardiovascular agents (e.g., beta blockers and angiotensin converting enzyme inhibitors); phosphodiesterase inhibitors; antivirals; antibiotics; antifungals; and antineoplastics.

More specific drugs that act on the CNS include, but are not limited to, SSRIs, benzodiazepine compounds, tricyclic antidepressants, antipsychotic agents, anti-anxiolytic agents, β-adrenergic antagonists, 5-HT_{1A} receptor antagonists, and 5-HT₃ receptor agonists. Even more specific drugs that act on the CNS include, but are not limited to, lorazepam, tomoxetine, olanzapine, respiradone, buspirone, hydroxyzine, and valium.

Examples of 5-HT₃ antagonists that can be used in compositions and methods of the invention include, but are not limited to, granisetron (KYTRIL®), metoclopramide (REGLAN@), ondansetron (ZOFRAN®), renzapride, zacopride, tropisetron, and optically active stereoisomers, active metabolites, and pharmaceutically acceptable salts, solvates, hydrates, clathrates, prodrugs, optically and pharmacologically active stereoisomers, and pharmacologically active metabolites thereof. Preferred 5-HT₃ antagonists are antiemetic agents.

Selective serotonin reuptake inhibitors are compounds that inhibit the central nervous system uptake of serotonin while having reduced or limited affinity for other neurologically active receptors. Examples of SSRIs include, but are not limited to, citalopram (CELEXA®); fluoxetine (PROZAC®); fluvoxamine (LUVOX®); paroxetine (PAXIL®); sertraline (ZOLOFT®); venlafaxine (EFFEXOR®); and pharmaceutically acceptable salts, solvates, hydrates, clathrates, prodrugs, optically and pharmacologically active stereoisomers, and pharmacologically active metabolites thereof.

Disorders that can be treated or prevented using a 2-hydroxy derivative of sibutramine or a sibutramine metabolite, or a pharmaceutically acceptable salt, solvate, hydrate, clathrate, or prodrug thereof, in combination with an SSRIs include, but are not limited to, depression, affective disorders, anxiety, eating disorders, and cerebral function disorders such as those described herein.

Benzodiazepine compounds that can be used in the methods and compositions of the invention include, but are not limited to, those described in Goodman & Gilman, The Pharmacological Basis of Therapeutics, 362-373 (9^{th} ed. McGraw-Hill, 1996). Examples of specific benzodiazepines include, but are not limited to, alprazolam, brotizolam, chlordiazepoxide, clobazam, clonazepam, clorazepate, demoxepam, diazepam, estazolam, flumazenil, flurazepam, halazepam, lorazepam, midazolam, nitrazepam, nordazepam, xazepam, prazepam, quazepam, temazepam, triazolam, pharmacologically active etabolites and stereoisomers thereof, and pharmaceutically acceptable salts, solvates, hydrates, esters, clathrates, and prodrugs thereof. The tradenames of some of these compounds are provided below.

The clinician, physician, or psychiatrist will appreciate which of the above compounds can be used in combination with a 2-hydroxy derivative of sibutramine or a sibutramine metabolite, or a pharmaceutically acceptable salt, solvate, hydrate, clathrate, or prodrug thereof, for the treatment or prevention of a given disorder, although preferred combinations are disclosed herein.

Disorders that can be treated or prevented using a 2-hydroxy derivative of sibutramine or a sibutramine metabolite, or a pharmaceutically acceptable salt, solvate, hydrate, clathrate, or prodrug thereof, in combination with a benzodiazepine such as those listed above include, but are not limited to, depression, affective disorders, anxiety, eating disorders, and cerebral function disorders such as those described herein.

The invention further encompasses methods of using and pharmaceutical compositions comprising 2-hydroxy derivative of sibutramine or a sibutramine metabolite, or a pharmaceutically acceptable salt, solvate, hydrate, clathrate, or prodrug thereof, in combination with an antipsychotic agent. Antipsychotic agents are used primarily in the management of patients with psychotic or other serious psychiatric illness marked by agitation and impaired reasoning. These drugs have other properties that possibly are useful clinically, including antiemetic and antihistamine effects and the ability to potentiate analgesics, sedatives, and general anesthetics. Specific antipsychotic drugs are tricyclic antipsychotic drugs, of which there are three subtypes: phenothiazines, thioxanthenes, and other heterocyclic compounds, all of which can be used in the methods and compositions of the invention. See, e.g., Goodman & Gilman, The Pharmacological Basis of Therapeutics, 404 (9^{th} ed. McGraw-Hill, 1996).

Specific tricyclic antipsychotic compounds include, but are not limited to, chlorpromazine, mesoridazine, thioridazine, acetophenazine, fluphenazine, perphenazine, trifluoperazine, chlorprothixene, thiothixene, clozapine, haloperidol, loxapine, molindone, pimozide, risperidone, desipramine, and pharmaceutically acceptable salts, solvates, hydrates, clathrates, prodrugs, optically and pharmacologically active stereoisomers, and pharmacologically active metabolites thereof. The tradenames of some of these compounds are provided herein.

Disorders that can be treated or prevented using a 2-hydroxy derivative of sibutramine or a sibutramine metabolite, or a pharmaceutically acceptable salt, solvate, hydrate, clathrate, or prodrug thereof, in combination with an antipsychotic compound, and particularly a tricyclic antipsychotic compound, include, but are not limited to, affective disorders (e.g., depression), anxiety, eating disorders, and cerebral function disorders (e.g., schizophrenia) such as those described herein.

The invention further encompasses methods of using and pharmaceutical compositions comprising a 2-hydroxy derivative of sibutramine or a sibutramine metabolite, or a pharmaceutically acceptable salt, solvate, hydrate, clathrate, or prodrug thereof, in combination with a non-benzodiazepine or non-tricyclic agents. Examples of such additional pharmacologically active compounds include, but are limited to: olanzapine, buspirone, hydroxyzine, tomoxetine, and pharmaceutically acceptable salts, solvates, hydrates, clathrates, prodrugs, optically and pharmacologically active stereoisomers, and pharmacologically active metabolites thereof.

Chlorpromazine, which is chemically named 10-(3-dimethylaminopropyl)-2-chlorphenothiazine, is sold under the tradename THORAZINE@. THORAZINE® is indicated, inter alia, for the management of manifestations of psychotic disorders. Physician's Desk Reference® 3101-3104 (53^{rd} ed., 1999).

The besylate salt of mesoridazine, which is chemically named 10-[2(1-methyl-2-piperidyl)ethyl]-2-methyl-sylfinyl)-phenothiazine, is sold under the tradename SERENTL®. SERENTIL® is indicated in the treatment of schizophrenia, behavioral problems in mental deficiency and chronic brain syndrome, alcoholism, and psychoneurotic manifestations. Physician 's Desk Reference® 764-766 (53^{rd} ed., 1999).

Perphenazine, which is chemically named 4-[3-(2-chlorophenothiazin-10-yl)propyl-1-piperazineethanol, is sold under the tradename TRILAFON®. TRILAFON® is indicated for use in the management of the manifestations of psychotic disorders and for the control of severe nausea and vomiting in adults. Physician 's Desk Reference® 2886-2888 (53^{rd} ed., 1999).

Trifluoperazine, which is chemically named 10-[3-(4-methyl-1-piperazinyl)-propyl]-2-(trifluoromethyl)-10H-phenothiazine, is sold under the tradename STELAZINE®. STELAZINE® is indicated for the management of the manifestations of psychotic disorders and for the short-term treatment of generalized non-psychotic anxiety. Physician 's Desk Reference® 3092-3094 (53^{rd} ed., 1999).

Thiothixene, which is chemically named N,N-dimethyl-9-[3-(4-methyl-1-piperazinyl)-propylidene]thioxanthene-2-sulfonamide, is sold under the tradename NAVANE®. NAVANE® is indicated in the management of manifestations of psychotic disorders. Physician's Desk Reference® 2396-2399 (53^{rd} ed., 1999).

Clozapine, which is chemically named 8-chloro-11-(4-methyl-1-piperazinyl)5H-dibenzo[b,e][1,4]diazepine, is sold under the tradename CLOZARIL®. CLOZARIL® is indicated for the management of severely ill schizophrenic patients who fail to respond adequately to standard antipsychotic drug treatment. Physician's Desk Reference® 2004-2009 (53^{rd} ed., 1999).

Haloperidol, which is chemically named 4-[4-(p-chlorophenyl)-4-hydroxy-piperidonol-4′-fluorobutyrophenone, is sold under the tradename HALDOL®. HALDOL® is indicated for use in the management of patients requiring prolonged parenteral antipsychotic therapy (e.g., patients with chronic schizophrenia). Physician 's Desk Reference® 2190-2192 (53^{rd} ed., 1999).

Loxapine, which is chemically named 2-chloro-11-(4-methyl-1-piperazinyl)dibenz[b,f][1-4]oxaxepine, is sold under the tradename LOXITANE®. LOXITANE® is indicated for the management of the manifestations of psychotic disorders. Physician 's Desk Reference® 3224-3225 (53^{rd} ed., 1999).

Molindone, which is chemically named 3-ethyl-6,7-dihydro-2-methyl-5-(morpholinomethyl) indol-4(5H)-one hydrochloride, is sold under the tradename MOBAN®. MOBAN® is indicated for the management of the manifestations of psychotic disorders. Physician's Desk Reference® 978-979 (53^{rd} ed., 1999).

Pimozide, which is chemically named, 1-[1-[4,4-bis(4-fluorophenyl)butyl]4-piperidinyl]-1,3-dihydro-2H-benzimidazole-2-one, is sold under the tradename ORAP®. ORAP® is indicated for the suppression of motor and phonic tics in patients with Tourette's Disorder who have failed to respond satisfactorily to standard treatment. Physician 's Desk Reference® 1054-1056 (53^{rd} ed., 1999).

Risperidone, chemically named 3-[2-[4-(6-fluoro-1,2-benzisoxazol-3-yl)-1-piperidinyl]ethyl]-6,7,8,9-tetrahydro-2-methyl-4H-pyrido[1,2-a]pyrimidin-4-one, is sold under the tradename RISPERDAL®. RISPERDAL® is indicated for the management of the manifestations of psychotic disorders. Physician's Desk Reference® 1432-1436 (53^{rd} ed., 1999).

The hydrochloride salt of desipramine, which is chemically named 5H-Dibenz[bf]azepine-5-propanamine-10,11-dihydro-N-methyl-monohydrochloride, is sold under the tradename NORPRAMIN®. NORPRAMIN® is indicated for the treatment of depression. Physician's Desk Reference® 1332-1334 (53^{rd} ed., 1999).

Olanzapine, which is chemically named 2-methyl-4-(4-methyl-1-piperazinyl)-10H-thieno[2,3-b][1,5]benzodiazepine, is sold under the tradename ZYPREXA®. ZYPREXA® is indicated for the management of the manifestations of psychotic disorders. Physician's Desk Reference® 1641-1645 (53^{rd} ed., 1999).

The hydrochloride salt of buspirone, which is chemically named 8-[4-[4-(2-pyrimidinyl)-1-piperazinyl]butyl]-8-azaspiro-[4.5]decane-7,9-dione monohydrochloride, is sold under the tradename BUSPAR®. BUSPAR® is indicated for the management of anxiety disorders or the short-term relief of the symptoms of anxiety. Physician 's Desk Reference® 823-825 (53^{rd} ed., 1999).

The hydrochloride salt of hydroxyzine, which is chemically named 1-(p-chlorobenzhydryl)-4[2-(2-hydroxyethoxy)-ethyl] piperazine dihydrochloride, is sold under the tradename ATARAX®. ATARAX® is indicated for symptomatic relief of anxiety and tension associated with psychoneurosis and as an adjunct in organic disease states in which anxiety is manifested. Physician 's Desk Reference® 2367-2368 (53^{rd} ed., 1999).

Disorders that can be treated or prevented using a racemic or stereomerically pure 2-hydroxy derivative of sibutramine or a sibutramine metabolite, or a pharmaceutically acceptable salt, solvate, or clathrate thereof, in combination with an antipsychotic compound, and particularly a tricyclic antipsychotic compound, include, but are not limited to, affective disorders (e.g., depression), anxiety, eating disorders, and cerebral function disorders (e.g., schizophrenia) such as those described herein.

Disorders that can be treated or prevented using a racemic or stereomerically pure 2-hydroxy derivative of sibutramine or a sibutramine metabolite, or a pharmaceutically acceptable salt, solvate, or clathrate thereof, in combination with a compound selected from the group consisting of lorazepam, tomoxetine, olanzapine, respiradone, buspirone, hydroxyzine, valium, pharmacologically active metabolites and stereoisomers thereof, and pharmaceutically acceptable salts, solvates, clathrates thereof include, but are not limited to, anxiety, depression, hypertension, and attention deficit disorders.

The invention further encompasses methods of using and pharmaceutical compositions comprising a racemic or stereomerically pure 2-hydroxy derivative of sibutramine or a sibutramine metabolite, or a pharmaceutically acceptable salt, solvate, or clathrate thereof, in combination with a 5-HT_{1A} receptor antagonist and/or a β-adrenergic antagonist. Examples of 5-HT_{1A} receptor antagonists and β-adrenergic antagonists that can be used in the methods and compositions of the invention include, but are limited to: alprenolol; WAY 100135; spiperone; pindolol; (S)-UH-301; penbutolol; propranolol; tertatolol; a compound of the formula I as disclosed in U.S. Pat. No. 5,552,429, which is incorporated herein by reference; and pharmaceutically acceptable salts, solvates, hydrates, clathrates, prodrugs, optically and pharmacologically active stereoisomers, and pharmacologically active metabolites thereof.

Alprenolol, which is chemically named 1-(1-methylethyl)amino-3-[2-(2-propenyl)phenoxy]-2-propanol, is described by U.S. Pat. No. 3,466,325, which is incorporated herein by reference.

WAY 100135, which is chemically named N-(t-butyl)-3-[4-(2-methoxphenyl)-piperazin-1-yl]-2-phenylpropanamide, is described by U.S. Pat. No. 4,988,814, which is incorporated herein by reference. See also, Cliffe et al., J. Med. Chem., 36:1509-1510 (1993).

Spiperone, which is chemically named 8-[4-(4-fluorophenyl)-4-oxobutyl]-1-phenyl-1,3,8-triazaspiro[4,5]decan-4-one), is described by U.S. Pat. Nos. 3,155,669 and 3,155,670, both of which are incorporated herein by reference. See also, Middlmiss et al., Neurosci. and Biobehav. Rev., 16:75-82 (1992).

Pindolol, which is chemically named 4-(2-hydroxy-3-isopropylaminopropoxy)-indole, is described by U.S. Pat. No. 3,471,515, which is incorporated herein by reference. See also, Dreshfield et al., Neurochem. Res., 21(5):557-562 (1996).

(S)-UH-301, which is chemically named (S)-5-fluoro-8-hydroxy-2-dipropylamino-tetralin), is well known to pharmacologists and pharmaceutical chemists. See, e.g., Hillyer et al., J. Med. Chem., 33:1541-44 (1990) and Moreau et al., Brain Res. Bull., 29:901-04 (1992).

Penbutolol, which is chemically named (1-(t-butylamino)-2-hydroxy-3-(2-cyclopentyl-phenoxy)propane), is sold under the tradename LEVATOL®. LEVATOL® is indicated the treatment of mild to moderate arterial hypertension. Physician 's Desk Reference® 2908-2910 (53^{rd} ed., 1999).

The hydrochloride salt of propranolol, which is chemically named 1-isopropylamino-3-(1-naphthalenyloxy)-2-propanol hydrochloride, is sold under the tradename INDERAL®. INDERAL® is indicated in the management of hypertension. Physician's Desk Reference® 3307-3309 (53^{rd} ed., 1999).

Tertatolol, chemically named 8-(3-t-butylamino-2-hydroxypropyloxy)-thiochroman, is described by U.S. Pat. No. 3,960,891, which is incorporated herein by reference.

Disorders that can be treated or prevented using a racemic or stereomerically pure 2-hydroxy derivative of sibutramine or a sibutramine metabolite, or a pharmaceutically acceptable salt, solvate, or clathrate thereof, in combination with a 5-HT_{1A} receptor antagonist include, but are not limited to, depression, obsessive-compulsive disorders, eating disorders, hypertension, migraine, essential tremor, hypertrophic subaortic stenosis and pheochromocytoma. A specific disorder that can be treated or prevented is posttraumatic depression disorder.

Disorders that can be treated or prevented using a racemic or stereomerically pure 2-hydroxy derivative of sibutramine or a sibutramine metabolite, or a pharmaceutically acceptable salt, solvate, or clathrate thereof, in combination with a β-adrenergic antagonist include, but are not limited to, post myocardial infarction depression.

The invention further encompasses methods of using and pharmaceutical compositions comprising a racemic or stereomerically pure 2-hydroxy derivative of sibutramine or a sibutramine metabolite, or a pharmaceutically acceptable salt, solvate, or clathrate thereof, in combination with a phosphodiesterase inhibitor. Examples of phosphodiesterase inhibitors that can be used in compositions and methods of the invention include, but are not limited to, those disclosed in U.S. Pat. Nos. 5,250,534; 5,719,283; 6,127,363; WO 94/28902; WO 97/03675; WO 98/06722, all of which are expressly incorporated herein by reference in their entirety. Preferred phosphodiesterase inhibitors are PDE5 and PDE6 inhibitors. Particular phosphodiesterase inhibitors include, but are not limited to, sildenophil (Viagra®), desmethylsildenophil, vinopocetine, milrinone, amrinone, pimobendan, cilostamide, enoximone, peroximone, vesnarinone, rolipran, R020-1724, zaprinast, dipyridamole, and pharmaceutically acceptable salts, solvates, hydrates, clathrates, prodrugs, optically and pharmacologically active stereoisomers, and pharmacologically active metabolites thereof.

Disorders and conditions that can be treated or prevented using a 2-hydroxy derivative of sibutramine or a sibutramine metabolite, or a pharmaceutically acceptable salt, solvate, hydrate, clathrate, or prodrug thereof, in combination with a phosphodiesterase include, but are not limited to, sexual dysfunction and cerebral function disorders. Others disorders and conditions include, but are not limited to, pain, migraines, osteoarthritis, and restless leg syndrome.

While all combinations of racemic and stereomerically pure 2-hydroxy derivatives of sibutramine metabolites and pharmaceutically acceptable salts, solvates, and clathrate thereof, and one or more of the above-described pharmacologically active compounds can be useful and valuable, certain combinations are particularly preferred. Examples of preferred combinations include those wherein a racemic or stereomerically pure 2-hydroxy derivative of sibutramine or a sibutramine metabolite, or a pharmaceutically acceptable salt, solvate, clathrate, or prodrug thereof, is combined with one of the following:

| | | |
|---|---|---|
| alprazolam; | halazepam; | mesoridazine; |
| brotizolam; | lorazepam; | thioridazine; |
| chlordiazepoxide; | midazolam; | acetophenazine; |
| clobazam; | nitrazepam; | fluphenazine; |
| clonazepam; | nordazepam; | perphenazine; |
| clorazepate; | oxazepam; | trifluoperazine; |
| demoxepam; | prazepam; | chlorprothixene; |
| diazepam; | quazepam; | thiothixene; |
| estazolam; | temazepam; | clozapine; |
| flumazenil; | triazolam; | haloperidol; |
| flurazepam; | chlorpromazine; | loxapine; |
| molindone; | desipramine; | amrinone; |
| pimozide; | clonidine; | pimobendan; |
| risperidone; | olanzapine; | cilostamide; |
| alprenolol; | methylphenidate; | enoximone; |
| WAY 100135; | buspirone; | peroximone; |
| spiperone; | hydroxyzine; | vesnarinone; |
| S(S)-pindolol; | tomoxetine; | rolipran; |
| R(R)-pindolol; | sildenophil; | R020-1724; |
| racemic pindolol; | desmethylsildenophil; | zaprinast; or |
| (S)-UH-301; | vinopocetine; | dipyridamole. |
| penbutolol; | milrinone; | |
| tertatolol; | | |

Suitable daily dosage ranges of additional pharmacologically active compounds that can be adjunctively administered with 2-hydroxy derivative of sibutramine or a sibutramine metabolite can be readily determined by those skilled in the art following dosages reported in the literature and recommended in the Physician's Desk Reference® (54^{th} ed., 2000).

For example, suitable daily dosage ranges of 5-HT₃ antagonists can be readily determined by those skilled in the art and will vary depending on factors such as those described herein and the particular 5-HT₃ antagonists used. In general, the total daily dose of a 5-HT₃ antagonist for the treatment or prevention of a disorder described herein is from about 0.5 mg to about 500 mg, preferably from about 1 mg to about 350 mg, and more preferably from about 2 mg to about 250 mg per day.

Similarly, suitable daily dosage ranges of phosphodiesterase inhibitors can be readily determined by those skilled in the art. In general, the total daily dose of a phosphodiesterase inhibitor will be from about 0.5 mg to about 500 mg, from about 1 mg to about 350 mg, or from about 2 mg to about 250 mg.

The therapeutic or prophylactic administration of an active ingredient of the invention (e.g., 2-hydroxy derivatives of sibutramine metabolites and additional pharmacologically active compounds) is preferably initiated at a lower dose and increased, if necessary, up to the recommended daily dose as either a single dose or as divided doses, depending on the global response of the patient. An example of a lower dose of 2-hydroxy derivative of sibutramine or a sibutramine metabolite is from about 0.1 mg to about 1 mg; an example of a lower dose of 5-HT₃ antagonist is from about 15 mg to about 60 mg. It is further recommended that patients aged over 65 years should receive doses of 2-hydroxy derivative of sibutramine or a sibutramine metabolite in the range of from about 0.1 mg to about 10 mg per day depending on global response. It may be necessary to use dosages outside these ranges, which will be readily determinable by one of ordinary skill in the pharmaceutical arts.

The dosage amounts and frequencies provided above are encompassed by the terms “therapeutically effective,” “prophylactically effective,” and “therapeutically or prophylactically effective” as used herein. When used in connection with an amount of a racemic or stereomerically pure 2-hydroxy derivative of sibutramine or a sibutramine metabolite, these terms further encompass an amount of racemic or stereomerically pure 2-hydroxy derivative of sibutramine or a sibutramine metabolite that induces fewer or less sever adverse effects than are associated with the administration of racemic sibutramine. Adverse effects associated with racemic sibutramine include, but are not limited to, significant increases in supine and standing heart rate, including tachycardia, increased blood pressure (hypertension), increased psychomotor activity, dry mouth, dental caries, constipation, hypohidrosis, blurred or blurry vision, tension, mydriasis, seizures, formation of gallstones, renal/hepatic dysfunction, fevers, arthritis, agitation, leg cramps, hypertonia, abnormal thinking, bronchitis, dyspnea, pruritus, amblyopia, menstrual disorder, ecchymosis/bleeding disorders, interstitial nephritis, and nervousness. See, e.g., Physician 's Desk Reference® 1494-1498 (53^{rd} ed., 1999). However, the induction of fewer or less severe adverse-effects is attributable to the administration of a 2-hydroxy derivative of sibutramine or a sibutramine metabolite and the efficacy of which may be less apparent or absent with the administration of a combination therapy.

### 4.3. Pharmaceutical Compositions

The invention encompasses pharmaceutical compositions and single unit dosage forms comprising a 2-hydroxy derivative of sibutramine or a sibutramine metabolite, or a pharmaceutically acceptable salt, solvate, hydrate, clathrate, or prodrug thereof. Preferred 2-hydroxy derivatives are stereomerically pure. Certain pharmaceutical compositions and unit dosage forms further comprise at least one additional pharmacologically active compound.

The pharmaceutical compositions and dosage forms of this invention are particularly useful in the methods herein, and may be suitable for oral, mucosal (e.g., nasal, sublingual, buccal, rectal, and vaginal), parenteral (e.g., intravenous, intramuscular or subcutaneous), or transdermal administration.

Preferred pharmaceutical compositions and dosage forms comprise a 2-hydroxy derivative of sibutramine or a sibutramine metabolite, or a pharmaceutically acceptable salt, solvate, hydrate, clathrate, or prodrug thereof in an amount from about 0.1 mg to about 60 mg, preferably from about 2 mg to about 30 mg, and more preferably from about 5 mg to about 15 mg. Pharmaceutical compositions and dosage forms of the invention typically also comprise one or more pharmaceutically acceptable excipients or diluents.

Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of disorder may contain larger amounts of one or more of the active ingredients it comprises than a dosage form used in the chronic treatment of the same disorder. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients, such as, desmethylsibutramine and didesmethylsibutramine and its optically active enantiomers in particular, can be accelerated by some excipients such as lactose, or when exposed to water. Active ingredients that comprise primary or secondary amines are particularly susceptible to such accelerated decomposition. Consequently, this invention encompasses pharmaceutical compositions and dosage forms that contain little, if any, lactose or mono- or di-saccharides. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmocopia (USP) SP (XXI)/NF (XVI). In general, lactose-free compositions comprise active ingredients, a binder/filler, and a lubricant in pharmaceutically compatible and pharmaceutically acceptable amounts. Preferred lactose-free dosage forms comprise active ingredients, microcrystalline cellulose, pre-gelatinized starch, and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine (e.g., desmethylsibutramine and didesmethylsibutramine) are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise a racemic or stereomerically pure 2-hydroxy derivative of sibutramine or a sibutramine metabolite, or a pharmaceutically acceptable salt, solvate, clathrate, hydrate, or prodrug thereof in an amount of from about 0.1 mg to about 60 mg, preferably in an amount of from about 2 mg to about 30 mg, and more preferably in an amount of from about 5 mg to about 15 mg.

### 4.3.1. Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington 's Pharmaceutical Sciences, 18th ed., Mack Publishing, Easton Pa. (1990).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, fillers, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, PA), and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

The magnitude of a prophylactic or therapeutic dose of an active ingredient in the acute or chronic management of a disorder or condition will vary with the severity of the disorder or condition to be treated and the route of administration. The dose, and perhaps the dose frequency, will also vary according to age, body weight, response, and the past medical history of the patient. Suitable dosing regimens can be readily selected by those skilled in the art with due consideration of such factors.

### 4.3.2. Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### 4.3.3. Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

### 4.3.4. Transdermal, Topical, and Mucosal Dosage Forms

Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdernal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### 4.3.5. Kits

Typically, active ingredients of the invention are preferably not administered to a patient at the same time or by the same route of administration. This invention therefore encompasses kits which, when used by the medical practitioner, can simplify the administration of appropriate amounts of active ingredients to a patient.

A typical kit of the invention comprises a unit dosage form of a 2-hydroxy derivative of sibutramine or a sibutramine metabolite, or a pharmaceutically acceptable prodrug, salt, solvate, hydrate, or clathrate thereof, and a unit dosage form of an additional pharmacologically active compound. Examples of additional pharmacologically active compounds are disclosed herein.

Kits of the invention can further comprise devices that are used to administer the active ingredients. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### 5. EXAMPLES

The invention can be further understood by reference to the following examples. It will be apparent to those skilled in the art that many modifications, both to materials and methods, can be practiced without departing from the scope of this invention.

### 5.1. Example 1

### Asymmetric Synthesis of 2-Hydroxydidesmethylsibutramine

Synthesis of (R)-t-butanesulfinic acid 1-(4-chlorophenyl)-cyclobutylmethyleneamide: To a solution of 1-(4-chlorophenyl)-cyclobutanecarboxaldehyde (2.0 g, 10.4 mmol) in THF (25 mL) at room temperature was added Ti(OEt)₄ (22.5 mL, ˜20% in ethanol) and t-butylsulfinamide (1.2 g, 9.9 nmol). After stirring for 6-8 hours, as monitored by TLC for the disappearance of t-butylsulfinamide, the reaction mixture was poured to a brine solution (30 mL) at room temperature with stirring. The resulting suspension was filtered and filter cake was washed with EtOAc. The filtrate was then washed with brine, dried over Na₂SO₄ and evaporated to provide the crude sulfinenamine product (2.9 g) with 98% yield (the product was used in the next step without purification). ¹H NMR (CDCl₃): 6 1.24 (s, 9H), 1.87-2.20 (m, 2H), 2.45-2.90 (m, 4H), 7.08-7.46 (m, 4H), 8.07 (s, 1H). ¹³C NMR (CDCl₃): δ 16.2, 22.6, 31.0, 31.4, 52.1, 57.3, 127.8, 128.9, 132.7, 142.7, 170.8. 3-[1-(4-Chloro-phenyl)-cyclobutyl]-3-(2-methyl-propane-2-sulfinylamino)-propionic acid ethyl ester: To a solution of lithium bis(trimethylsilyl)amide (24.3 mL, 24.3 mmol) in dry tetrahydrofuran (60 mL) at −78° C. was slowly added ethyl acetate (2.37 mL, 24.2 mmol). After stirring at −78° C. for 20 minutes, the solution was warmed to −40° C. over 10 minutes. A solution of 2-Methyl-propane-2-sulfinic acid 1-(4-chloro-phenyl)-cyclobutylinethyleneamide (5.57 g, 18.7 inmol) in THF (10 mL) was added to it −40° C. and the resulting mixture was stirred for 1 hour. The reaction mixture was quenched with aqueous NaHCO₃ solution (5 mL) and warmed to room temperature. Ethyl acetate (60 mL) and aqueous NaHCO₃ solution (60 mL) was added, and the layers were separated. The aqueous layer was washed with EtOAc (60 mL), and the combined organic layers were dried (MgSO₄), filtered, and concentrated in vacuo. The crude oil was purified by column chromatography using 28-35% EtOAc/hexane as eluent to provide 2.55 g (26%) yield of the less polar product (R,R)-diastereoisomer and 5.63 g (58%) of the more polar product (R,S)-diastereoisomer.

¹H NMR (CDCl₃) of the less polar product [(R,R-diastereoisomer)]: δ 1.10 (s, 9H), 1.22 (t, J=6.7 Hz, 3H), 1.81 (m, 2H), 2.03 (m, 1H), 2.24 (m, 1H), 2.45 (m, 2H), 2.50 (dd, J=4.0, 14.0 Hz, 1H), 2.78 (m, 1H), 3.02 (d, J=9.8 Hz, 1H), 4.06 (m, 3H), 7.22 (d, J=8.7 Hz, 2H), 7.35 (d, J=8.7 Hz, 2H).

¹H NMR (CDCl₃) of the more polar product [(R,S)-diastereoisomer)]: δ 1.24 (m, 12H), 1.85 (m, 1H), 2.02-2.42 (m, 6H), 2.60 (dd, J 4.2, 15.7 Hz, 1H), 3.67 (d, J=10.0 Hz, 1H), 4.03 (m, 3H), 7.08 (d, J=8.5 Hz, 2H), 7.28 (d, J=8.5 Hz, 2H). M⁺385.7. 30

3-[1-(4-Chloro-phenyl)-cyclobutyl]-3-(2-methyl-propane-2-sulrlnylamino)-propionic acid ethyl ester (via titanium enolate): To a solution of LDA (0.625 mL, 1.25 inmol) in dry tetrahydrofuran (4 mL) at −78° C. was slowly added ethyl acetate (0.117 mL, 1.20 mmol). After stirring at −78° C. for 30 minutes, a solution of ClTi(iOPr)₃ (0.621 mL, 2.60 mmol) in THF (2.0 mL) was added at −78° C. The reaction stirred for 40 mm at −78° C., then 2-Methyl-propane-2-sulfinic acid 1-(4-chloro-phenyl)-cyclobutylmethyleneamide (0.30 g, 1.0 mmol) in THF (3 mL) was added at −78° C. and stirred for 3 hours. The reaction mixture was quenched with aqueous NaHCO₃ solution (5 mL) and warmed to room temperature. Ethyl acetate (60 mL) and aqueous NaHCO₃ solution (60 mL) was added, and the layers were separated. The aqueous layer was washed with EtOAc (60 mL), and the combined organic layers were dried (MgSO₄), filtered, and concentrated in vacuo to provide crude product showing 91:9 selectivity for the (R,S)-stereoisomer. ¹H NMR spectra are identical to the product resulting from the lithium enolate.

Synthesis of (R,R)-2-Methylpropane-2-sulfinic acid {1-11-(4-chlorophenyl)-cyclobutyl]-3-hydroxy-3-methyl-butyl}-amide from (R,R) diastereoisomer: 3-[1-(4-chlorophenyl)-cyclobutyl]-3-(2-methylpropane-2-sulfinylamino)-propionic acid ethyl ester (less polar (R,R)-isomer, 2.9 g) was dissolved in anhydrous THF (30 mL), and was added to a solution of MeMgBr (Aldrich 3 M in Ether, 14 mL) in THF (20 mL) at −78° C. The reaction mixture was warmed to room temperature for 30 minutes, followed by heating to 60° C. for 1.5 hours. The reaction mixture was then cooled to 0° C. and quenched with water (30 mL), extracted with ethyl acetate (60 mL). The extract was washed with water (10 mL), brine (20 mL), and concentrated to give an oil (crude product). This crude product was dissolved in MeOH (30 mL), and added sodium borohydride (1.0 g) at room temperature. The reaction mixture was stirred for 20 minutes, concentrated to remove MeOH to give a white solid. It was quenched with water (20 mL), and extracted with ethyl acetate. The extract was washed with water, brine and concentrated to give a crude product (1.2 g). It was passed through a silica gel column (EtOAc:hexane =1:1) to give the product (1.07 g) as white solids. ¹H NMR(CDCl₃) δ 1.19 (s, 9H), 1.20 (s, 3H), 1.25(s, 3H), 1.20-1.30 (m, 1H), 1.65 (dd, J1=1.5 Hz, J2=15 Hz, 1H), 1.70-1.90 (m, 2H), 2.13-2.25 (M 1H), 2.35-2.52 (m, 2H), 2.60-2.72 (m, 1H), 2.95 (s, 1H), 3.33 (d, J=9.9 Hz, 1H), 3.74 (t, d J1=1.3 Hz, J2=10.2 Hz), 7.35 (s, 4H). ¹³C δ 15.1, 22.9, 28.7, 31.2, 32.5, 35.0, 42.9, 50.9, 56.7, 62.7, 70.2, 127.9, 130.4, 132.2, 141.7. M⁺ 371.9.

Synthesis of (R,S)-2-Methylpropane-2-sulfinic acid {1-[1-(4-chlorophenyl)-cyclobutyl]-3-hydroxy-3-methyl-butyl}-amide from (R,S) diastereoisomer: 3-[1-(4-chlorophenyl)-cyclobutyl]-3-(2-methylpropane-2-sulfinylamino)-propionic acid ethyl ester (more polar (R,S)-isomer, 3.3 g) was dissolved in anhydrous THF (30 mL), and was added to a solution of MeMgBr (Aldrich 3 M in Ether, 12 mL) in THF (20 mL) at −78° C. The reaction mixture was warmed to room temperature for 30 minutes, followed by heating to 50° C. for 2 hours. The reaction mixture was then cooled to 0° C. and quenched with water (30 mL), extracted with ethyl acetate (60 mL). The extract was washed with brine, concentrated to give a crude product (2.6 g), it was passed through a column of silica gel (EtOAC:hexane=7:3) to give the product as an oil (1.2 g). ¹H NMR (CDCl₃) δ 1.0-1.15 (m, 1H), 1.1 (s, 3H), 1.27 (s, 9H), 1.39 (s, 3H), 1.50 (d, J=13 Hz, 1H), 1.88-1.90 (m, 1H), 1.95-2.18 (m, 2H), 2.19-2.48 (m, 3H), 3.54 (d, J=8 Hz, 1H), 3.83 (m, 1H), 4.42 (s, 1H), 7.07 (d, J=8.4 Hz, 2H), 7.26 (d, J=8.4 Hz, 2H). ¹³C δ 14.8, 22.7, 26.9, 31.3, 32.1, 43.5, 50.9, 56.2, 59.2, 69.6, 127.4,128.8, 131.6,143.5. M⁺ 272.2.

Synthesis of (S)-(+)-4-amino-[1-(4-chlorophenyl)cyclobutyl]-2-methyl-butan-2-ol [(+)-2-OH-DDMS): 2-Methylpropane-2-sulfinic acid {1-[1-(4-chlorophenyl)-cyclobutyl]-3-hydroxy-3-methyl-butyl}-amide from (R,S)-diastereoisomer, (1.2 g) was dissolved in MeOH (15 mL) and added aqueous HCl (5 N, 25 mL). The reaction mixture was stirred for 2 hours at room temperature. The reaction mixture was concentrated to remove MeOH, and the residue was cooled to 0-4° C. and neutralized with 50% NaOH until basic. The aqueous solution was then extracted with dichloroethane (60 mL), and concentrated to give a crude product (0.9 g). The product was purified by flash chromatography (EtOAc:MeOH 8:2) to yield 0.59 g product. ¹H NMR (CDCl₃) δ 0.79-0.88 (t, J=11 Hz, 1H), 1.15 (s, 3H), 1.28 (s, 3H), 1.50-1.56 (1H, dd, J1=1.8 Hz, J2=14.1 Hz, 1.80-2.44 (m, 6H), 2.40-3.00 (broad s, 3H), 3.28-3.32 (1H, dd, J1=1.8 Hz, J2=12 Hz), 6.99-7.02 (2H, d, J=8.7 Hz), 7.28-7.31 (2H, d, J=8.4 Hz). ¹³ C δ 15.2, 28.4, 31.1, 31.3, 32.0, 42.3, 51.1, 55.9, 69.9, 127.9, 128.6, 131.9, 143.5. M⁺ 267.8. Optical rotation [α]: +5.3 (c 1, MeOH). Enantiomeric excess (98.4) was determined by HPLC. Chiral OD, 10 μm, 4.6×250 m, hexane/EtOH/MeOH/DEA (96:2:2:0.1). (R)-(−)-isomer 17.74 min., (S)-(+)-isomer 20.99 min.

Synthesis of (R)-(−)-4-amino-[1-(4-chlorophenyl)cyclobutyl]-2-methyl-butan-2-ol [(R)-(−)-2-OH-DDMS]: 2-Methylpropane-2-sulfinic acid (1-[1-(4-chlorophenyl)-cyclobutyl]-3-hydroxy-3-methyl-butyl}-amide from (R,R)-diastereoisomer, (1.1 g) was dissolved in THF (20 mL) and cooled to 10-15° C. To it was added aqueous HCl (5 N, 10 mL). The reaction mixture was stirred for 10 minutes at room temperature. The reaction mixture was concentrated to remove THF. The residue was added water (915 mL), and neutralized with 50% NaOH until basic and extracted with ethyl acetate (40 mL). The extract was washed with water (10 mL), brine, and concentrated to give the final product (0.77g). Enantiomer excess (ee) by HPLC (99.4). Optical rotation [α]: −5.3 (c 1, MeOH). ¹H and ¹³C NMR spectra are identical to its enantioiner. M⁺ 267.8.

Synthesis of (R)-2-OH-DDMS L-tartrate: To a solution of (R)-(−)-2-OH-DDMS (0.6 g) in MeOH (10 mL) was added a solution of L-tartaric acid (0.33 g) in water (2 mL). The reaction mixture was stirred for 10 minutes and concentrated to give a white solid, the product. ¹H NMR (DMSO-d₆). δ 1.1-1.2 (m, 1H), 1.12 (s, 3H), 1.17 (s, 3H), 1.56-1.59 (d, J=9 Hz, 1H), 1.65-1.80 (m, 1H), 1.85-1.98 (m, 1H), 2.22-2.30 (m, 3H), 5.50-5.70 (m, 1H), 3.65 (d, J=9 Hz, 1H), 3.9 (s, 2H), 5.50-7.00 (broad s, 7H), 7.26-7.29 (d, J 9 Hz, 2H), 7.40-7.43 (d, J 9 Hz, 2H).

Synthesis of {(S)-1-[1-(4-Chloro-phenyl)-cyclobutyl]-3-hydroxy-3-methyl-butyl}-carbamic acid ethyl ester: To a solution of (S)-(+)-2-OH-DDMS (0.644 g, 2.40 mmol, >99.9% ee) in CH₂Cl₂ (25 mL) was added N,N-diisopropylethyl amine (0.503 mL, 2.88 mmol) and ethyl chloroformate (0.24 mL, 2.52 mmol). The reaction mixture was stirred for 90 minutes, then quenched with aqueous 0.1 N HCl (50 mL). The layers were separated and the organic layer was washed with aqueous NaHCO₃ solution. The organic layer was dried (MgSO₄), filtered and concentrated in vacuo to provide 0.82 g of crude product (100%). δ 0.90 (dd, J=10.5 Hz, 14.4 Hz, 1H), 1.18 (s, 3H), 1.25 (s, 3H), 1.28 (m, 3H), 1.63 (d, J=14.4 Hz, 1H), 1.85 (m, 1H), 2.22 (m, 4H), 4.14 (m, 2H), 4.28 (t, J=10.4 Hz, 1H), 4.42 (d, J=10.4 Hz, 1H), 7.08 (d, J=8.4 Hz, 2H), 7.31 (d, J=8.4 Hz, 2H). M⁺ (—H₂O) 321.9.

Synthesis of (−)-4-11-(4-Chloro-phenyl)-cyclobutyl]-2-methyl-4-methylamino-butan-2-ol [(S)-(−)-2-OH-DMS]: A solution of crude (1-[1-(4-Chloro-phenyl)-cyclobutyl]-3-hydroxy-3-methyl-butyl}-carbamic acid ethyl ester (0.82 g) in THF (24.7 mL) was treated with LAH (0.44 g, 12.0 mmol) at room temperature. The reaction mixture was allowed to stir for 1.5 hours at 65° C. followed by cooling to 0° C. The reaction mixture was carefully quenched with water (100 mL). The layers were separated and the aqueous layer was washed with ethyl acetate (2×100 mL). The combined organic layers were dried (MgSO₄), filtered and concentrated in vacuo to provide 0.623g of crude product. The crude oil was purified by column chromatography using 0-20% MeOH/EtOAc as eluent to provide 623 mg (92%) yield of pure product (3 steps). Optical rotation [a]: -15.3 (c 1.04, MeOH). ¹H NMR (CDCl₃). δ 0.82 (dd, J=12.0, 13.9 Hz, 1H), 1.13 (s, 3H), 1.37 (dd, J=1.8, 14.0 Hz, 1H), 1.84 (m, 2H), 2.18 (q, J=9.1 Hz, 1H), 2.41 (m, 3H), 2.60 (s,3H), 3.11 (dd, J=2.0, 12.0 Hz, 1H), 7.23 (d, J=8.5 Hz, 2H), 7.38 (d, J=8.5 Hz, 2H). M⁺ 281.9.

Synthesis of {1-[1-(4-Chloro-phenyl)-cyclobutyl]-3-hydroxy-3-methyl-butyl}-carbamic acid ethyl ester: To a solution of (R)-(−)-2-OH-DDMS (0.68 g, 2.54 mmol, 99.8% ee) in CH₂Cl₂ (26.4 mL) was added N,N-diisopropylethyl amine (0.532 mL, 3.05 mmol) and ethyl chloroformate (0.25 mL, 2.67 mmol). The reaction mixture was stirred for 90 minutes, then the reaction was quenched with aqueous 0.1 N HCl (50 mL). The layers were separated and the organic layer was washed with aqueous NaHCO₃ solution. The organic layer was dried (MgSO₄), filtered and concentrated in vacuo to provide crude product. ¹H NMR (CDCl₃). δ 0.90 (dd, J=10.5, 14.4 Hz, 1H), 1.18 (s, 3H), 1.25 (s, 3H), 1.28 (m, 3H), 1.63 (d, J=14.4 Hz, 1H), 1.85 (m, 1H), 2.22 (m, 4H), 4.14 (m, 2H), 4.28 (t, J=10.4 Hz, 1H), 4.42 (d, J=10.4 Hz, 1H), 7.08 (d, J=8.4 Hz, 2H), 7.31 (d, J=8.4 Hz, 2H). M⁺ (—H₂O) 321.9.

Synthesis of (R)-(+)-4-[1-(4-Chloro-phenyl)-cyclobutyl]-2-methyl-4-methylamino-butan-2-ol [(R)-(+)-2-OH-DMS]: A solution of crude {1-[1-(4-Chloro-henyl)-cyclobutyl]-3-hydroxy-3-methyl-butyl}-carbamic acid ethyl ester in THF (26.1 mL) was treated with LAH (0.47 g, 12.7 mmol) at room temperature. The reaction mixture was allowed to stir for 1.5 hours at 65° C., followed by cooling to 0° C. The reaction mixture was carefully quenched with water (100 mL). The layers were separated and the aqueous layer was washed with ethyl acetate (2×100 mL). The combined organic layers were dried (MgSO₄), filtered and concentrated in vacuo to provide 0.623g of crude product. The crude oil was purified by column chromatography using 0-20% MeOH/EtOAc as eluent to provide 590 mg (82%) yield of pure product (3 steps). Optical rotation [α]: +15.1 (c 1.23, MeOH). ¹H and ¹³C NMR spectra are identical to its enantiomer. M⁺ 281.9.

Synthesis of (−)-4-[1-(4-Chloro-phenyl)-cyclobutyl]-2-methyl-4-methylamino-butan-2-ol (L)-Tartaric acid [(−)-OH-DMS-L-tartrate]: (−)-4-[1-(4-Chloro-phenyl)-cyclobutyl]-2-methyl-4-methylamino-butan-2-ol (0.86 g) was dissolved in 5 mL of methanol. To it at room temperature, was added a solution of L-tartaric acid in water (0.46 g, 3 nL H₂O). The reaction mixture was stirred for 1 hour. The solution was concentrated in vacuo to provide 1.32 g of a white foam (100%). ¹H NMR (CDCl₃). δ 1.19 (s, 3H), 1.54-1.91 (m, 5H), 2.44 (m, 3H), 2.55 (s, 3H), 3.45 (bs, 1H), 4.41 (bs, 2H), 7.37 (m, 4H), 7.67 (bs, 2H). ¹³C 6 16.2, 27.3, 31.8, 33.4, 38.9, 48.7, 65.5, 70.4, 72.8, 129.1, 133.2, 141.7, 176.4. M⁺ 281.9.

Synthesis of (+)-4-[1-(4-Chloro-phenyl)-cyclobutyl]-2-methyl-4-methylamino-butan-2-ol (L)-Tartaric acid [(+)-OH-DMS-L-tartrate]: (+)-4-[1-(4-Chloro-phenyl)-cyclobutyl]-2-methyl-4-methylamino-butan-2-ol (0.59 g) was dissolved in 5 mL of methanol. To it at room temperature, was added a solution of L-tartaric acid in water (0.31 g, 3 mL H₂O). The reaction mixture was stirred for 1 hour. The solution was concentrated in vacuo to provide 0.90 g of a white foam (100%). ¹H NMR (CDCl₃). δ 1.19 (s, 3H), 1.54-1.91 (m, 5H), 2.44 (m, 3H), 2.55 (s, 3H), 3.45 (bs, 1H), 4.41 (bs, 2H), 7.37 (m, 4H), 7.67 (bs, 2H). ¹³C δ 16.2, 27.3, 31.8, 33.4, 38.9, 48.7, 65.5, 70.4, 72.8, 129.1, 133.2, 141.7, 176.4. M⁺ 281.9.

### 5.2. Example 2

### In Vitro Activity of 2-Hydroxy Derivatives of Sibutramine Metabolites

The 2-hydroxy derivatives of the sibutramine metabolites N-desmethyl and N-didesmethylsibutramine (DMS and DDMS, respectively) were tested for their inhibition of functional uptake of serotonin (5-HT), norepinephrine (NE), or dopamine (DA), into synaptosomes prepared from rat whole brain, hypothalamus, or corpora striata, respectively, using methods known in the art. In addition, binding was determined at the nonselective muscarinic receptor and the p₃-receptor from rat cerebral cortex and rat adipose tissue, respectively.

Compounds were tested initially at three concentrations (10⁻⁹, 10⁻⁷, 10⁻⁵ M) and if ≧50% inhibition of uptake or binding was observed, they were tested further at ten different concentrations in duplicate in order to obtain full inhibition or competition curves. IC₅₀ values (concentration inhibiting control activity by 50 percent) were then determined by nonlinear regression analysis of the inhibition curves and tabulated below.

IC₅₀ Values (nM) for 2-OH Metabolites of DMS and DDMS
in Functional Uptake and Muscarinic Binding Assays

| | | | | Muscarinic |
|---|---|---|---|---|
| Compound | 5-HT | NE | DA | Binding |
| 2-(R)-OH-DMS | 22 | 23 | 48 | 8,970 |
| 2-(S)-OH-DMS | 5,400 | 310 | 370 | 4,480 |
| 2-(R)-OH-DDMS | 7.2 | 6.2 | 18 | — |
| 2-(S)-OH-DDMS | 670 | 71 | 59 | — |
| Imipramine | 23 | | | |
| Protriptyline | | 2 | | |
| GBR 12909 | | | 2.1 | |
| Atropine | | | | 0.38 |

—indicates <50% inhibition

IC₅₀ values for β₃-binding were not calculated because none of the compounds showed inhibition of greater than 50 percent; the maximum inhibition was 37 percent for 2-OH-(R)-DDMS.

It is clear from these results that enantiomerically pure (R)-didesmethylsibutramine is a potent serotonin reuptake inhibitor. Advantageously, however, muscarinic binding does not appear to be clinically relevant.

While the invention has been described with respect to the particular embodiments, it will be apparent to those skilled in the art that various changes and modifications may be made without departing from the spirit and scope of the invention as defined in the claims. Such modifications are also intended to fall within the scope of the appended claims.

What is claimed is:

1. A compound of Formula 1: 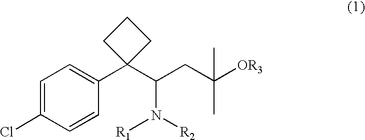
wherein each of R₁ and R₂ is independently hydrogen, substituted or unsubstituted alkyl, substituted or unsubstituted aryl, substituted or unsubstituted aralkyl, ketone, ester, or amide, and R₃ is hydrogen, substituted or unsubstituted alkyl, substituted or unsubstituted aryl, substituted or unsubstituted aralkyl, and pharmaceutically acceptable salts, solvates, hydrate, clathrates, and prodrugs thereof.

2. The compound of claim 1 wherein said compound is stereomerically pure.

3. 2-Hydroxysibutramine, or a pharmaceutically acceptable salt, solvate, hydrate, clathrate, or prodrug thereof.

4. The 2-hydroxysibutramine of claim 3 wherein said compound is stereomerically pure.

5. 2-Hydroxydesmethylsibutramine, or a pharmaceutically acceptable salt, solvate, hydrate, clathrate, or prodrug thereof.

6. The 2-hydroxydesmethylsibutramine of claim 5 wherein said compound is stereomerically pure.

7. 2-Hydroxydidesmethylsibutramine, or a pharmaceutically acceptable salt, solvate, hydrate, clathrate, or prodrug thereof.

8. The 2-hydroxydidesmethylsibutramine of claim 7 wherein said compound is stereomerically pure.

9. A method of preparing 2-hydroxydidesmethylsibutramine, or a pharmaceutically acceptable salt, solvate, clathrate, hydrate, or prodrug thereof, which comprises contacting a compound of Formula 2: 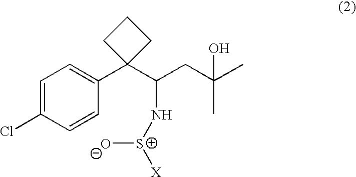
wherein X is substituted or unsubstituted alkyl, substituted or unsubstituted aralkyl, or substituted or unsubstituted aryl, with a reagent capable of cleaving a nitrogen-sulfur bond under conditions suitable for the formation of 2-hydroxydidesmethylsibutramine.

10. A method of preparing 2-hydroxydesmethylsibutramine, or a pharmaceutically acceptable salt, solvate, clathrate, hydrate, or prodrug thereof, which comprises contacting a compound of Formula 2 with a reagent capable of cleaving a nitrogen-sulfur bond under conditions suitable for the formation of 2-hydroxydidesmethylsibutramine, and contacting the 2-hydroxydidesmethylsibutramine with a methylating reagent under conditions sufficient for the formation of 2-hydroxydesmethylsibutramine.

11. The method of claim 9 or 10 wherein the compound of Formula 2 is prepared by contacting a compound of Formula 3: 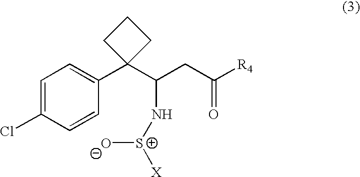
wherein R₄ is a substituted or unsubstituted alkyl (e.g., methyl, ethyl, or t-butyl), aryl, —OR₅, or —SR₅, wherein R₅ is alkyl, aryl, or aralkyl, with an alkylating agent under conditions sufficient for the formation of the compound of Formula 2.

12. The method of claim 11 wherein the compound of Formula 3 is prepared by contacting a compound of Formula 4: 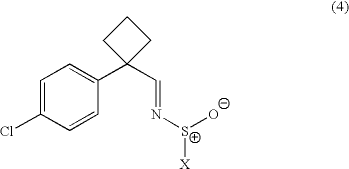
with a compound of Formula 5: 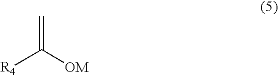
wherein M is a metal under conditions sufficient for the formation of the compound of Formula 3.

13. The method of claim 12 wherein the compound of Formula 4 is prepared by contacting a compound of Formula 6: 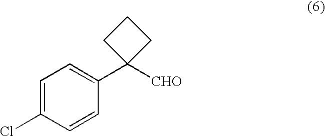
with a compound of Formula 7: 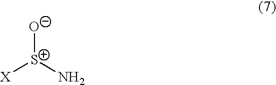
under conditions suitable for the formation of the compound of Formula 4.

14. A method of preparing 2-hydroxydidesmethylsibutramine, or a pharmaceutically acceptable salt, solvate, clathrate, hydrate, or prodrug thereof, which comprises contacting a compound of Formula 3 with a reagent capable of cleaving a nitrogen-sulfur bond under conditions suitable for the formation of the compound of Formula 8: 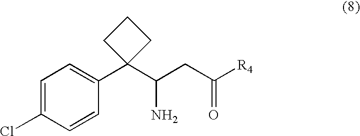
and contacting the compound of Formula 8 with an alkylating agent under conditions suitable for the formation of 2-hydroxydidesmethylsibutramine.

15. A method of preparing 2-hydroxydesmethylsibutramine, or a pharmaceutically acceptable salt, solvate, clathrate, hydrate, or prodrug thereof, which comprises contacting a compound of Formula 3 with an alkylating agent under conditions suitable for the formation of a compound of Formula 2, and contacting the compound of Formula 2 with a reagent capable of cleaving a nitrogen-sulfur bond under conditions suitable for the formation of 2-hydroxydidesmethyl-sibutramine, and contacting the 2-hydroxydidesmethylsibutramine with a methylating reagent under conditions sufficient for the formation of 2-hydroxydesmethylsibutramine.

16. A method of treating or preventing a sexual function disorder, which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof, optionally in combination with a 5-HT₃ antagonist.

17. A method of treating or preventing an affective disorder which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof.

18. The method of claim 17 wherein the affective disorder is depression, attention deficit disorder, a bipolar and manic condition, dysthymic disorder, or a cyclothymic disorder.

19. A method of treating or preventing weight gain or obesity which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof, optionally in combination with a lipase inhibitor.

20. A method of treating or preventing a disorder associated with the administration of a lipase inhibitor for obesity or weight management, which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof.

21. A method of treating or preventing a cerebral function disorder which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof.

22. The method of claim 21 wherein the cerebral function disorder is senile dementia, Alzheimer's type dementia, memory loss, amnesia/amnestic syndrome, disturbance of consciousness, coma, lowering of attention, speech disorders, Parkinson's disease, Lennox syndrome, autism, epilepsy, hyperkinetic syndrome, or schizophrenia.

23. A method of treating or preventing restless leg syndrome, which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof.

24. A method of treating or preventing pain which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof.

25. The method of claim 24 wherein the pain is acute or chronic neuropathic pain.

26. A method of treating or preventing obsessive-compulsive disorder which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof.

27. A method of treating or preventing substance abuse which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof.

28. A method of treating or preventing nicotine addiction which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof.

29. A method of eliciting smoking cessation which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof.

30. A method of treating or preventing a chronic disorder, which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof.

31. A method of treating or preventing anxiety which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof.

32. A method of treating or preventing an eating disorder including, but not limited to, anorexia, bulimia, binging, and snacking, which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof.

33. A method of treating or preventing migraines which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof.

34. A method of treating or preventing premenstrual syndrome, perimenopause,, or menopause, which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof.

35. A method of treating or preventing incontinence which comprises administering to a patient in need thereof a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof.

36. The method of claim 16, 17, 19-21, 23, 24, or 26-35 wherein the 2-hydroxy derivative of a sibutramine metabolite is 2-hydroxydesmethylsibutramine or 2-hydroxydidesmethylsibutramine.

37. The method of claim 36 wherein the 2-hydroxysibutramine or 2-hydroxy derivative of a sibutramine metabolite is stereomerically pure.

38. The method of claim 37 wherein the 2-hydroxy derivative of a sibutramine metabolite is enantiomerically pure (R)-2-hydroxydidesmethylsibutramine or (S)-2-hydroxydidesmethylsibutramine.

39. The method of claim 37 wherein the 2-hydroxy derivative of a sibutramine metabolite is enantiomerically pure (R)-2-hydroxydesmethylsibutramine or (S)-2-hydroxydesmethylsibutramine.

40. A pharmaceutical composition which comprises a therapeutically or prophylactically effective amount of 2-hydroxysibutramine or a 2-hydroxy derivative of a sibutramine metabolite, or a pharmaceutically acceptable salt, solvate, hydrate, clathrate, or prodrug thereof.

41. The pharmaceutical composition of claim 40 wherein said composition is lactose-free.

42. The pharmaceutical composition of claim 38 wherein said composition is suitable for oral, mucosal, parenteral, or transdermal administration to a patient.
